(19) United States
(12) Patent Application Publication
(10) Pub. No.: US 2022/0275380 A1
(43) Pub. Date: Sep. 1, 2022

(54) PLASMIDS

(71) Applicant: SNIPR Biome ApS, Copenhagen (DK)

(72) Inventor: Andreas PORSE, Copenhagen (DK)

(73) Assignee: SNIPR Biome ApS, Copenhagen (DK)

(21) Appl. No.: 17/637,414

(22) PCT Filed: Aug. 21, 2020
(86) PCT No.: PCT/EP2020/073512

§ 371 Date: Feb. 22, 2022

(30) Foreign Application Priority Data
Aug. 23, 2019 (GB) 1912176.3

(51) Int. Cl. C12N 15/70; C12N 15/11; A61K 35/74

(52) U.S. Cl. CPC C12N 15/70; C12N 15/11; A61K 35/74; C12N 2800/101

(57) ABSTRACT

The invention relates to means for carrying out conjugation between bacteria, and in particular the invention relates to carrier (ie, donor) bacteria comprising antimicrobial agents and methods of use. A carrier bacterium is capable of conjugative transfer of DNA encoding the agent to a target cell (ie, recipient cell). The invention provides novel plasmids that are devoid a functional hypC2 nucleotide sequence for these purposes.

### TECHNICAL FIELD

The invention relates to means for carrying out conjugation between bacteria, and in particular the invention relates to carrier (ie, donor) bacteria comprising antimicrobial agents and methods of use. A carrier bacterium is capable of conjugative transfer of DNA encoding the agent to a target cell (ie, recipient cell). The invention provides novel plasmids that are devoid a functional hypC2 nucleotide sequence for these purposes.

### BACKGROUND

DNA sequences controlling extra-chromosomal replication (ori) and transfer (tra) are distinct from one another; i.e., a replication sequence generally does not control plasmid transfer, or vice- versa. Replication and transfer are both complex molecular processes that make use of both plasmid- and host-encoded functions. Bacterial conjugation is the unidirectional and horizontal transmission of genetic information from one bacterium to another. The genetic material transferred may be a plasmid or it may be part of a chromosome. Bacterial cells possessing a conjugative plasmid contain a surface structure (the sex pilus) that is involved in the coupling of donor and recipient cells, and the transfer of the genetic information. Conjugation involves contact between cells, and the transfer of genetic traits can be mediated by many plasmids. Among all natural transfer mechanisms, conjugation is the most efficient. For example, F plasmid of E. coli, pCFlO plasmid of Enterococcus faecalis and pXO16 plasmid of Bacillus thuringiensis employ different mechanisms for the establishment of mating pairs, the sizes of mating aggregates are different, and they have different host ranges within gram-negative (F) as well as gram-positive (pCFlO and pXO16) bacteria. Their plasmid sizes are also different; 54, 100 and 200 kb, respectively. Remarkably, however, those conjugation systems have very important characteristics in common: they are able to sustain conjugative transfer in liquid medium and transfer efficiencies close to 100% are often reached in a very short time. Thus, the conjugative process permits the protection of plasmid DNA against environmental nucleases, and the very efficient delivery of plasmid DNA into a recipient cell. Conjugation functions are naturally plasmid encoded. Numerous conjugative plasmids (and transposons) are known, which can transfer associated genes within one species (narrow host range) or between many species (broad host range). Transmissible plasmids have been reported in numerous Gram-positive genera, including but not limited to pathogenic strains of Streptococcus, Staphylococcus, Bacillus, Clostridium and Nocardia. The early stages of conjugation generally differ in Gram-negative and Gram-positive bacteria. The role of some of the transfer genes in conjugative plasmids from Gram-negative bacteria are to provide pilus-mediated cell-to-cell contact, formation of a conjugation pore and related morphological functions. The pili do not appear to be involved in initiating conjugation in Gram-positive bacteria.

### SUMMARY OF THE INVENTION

The invention provides:

In a First Configuration

A conjugative plasmid that is devoid of a hypC2 nucleotide sequence or a homologue thereof.

In a Second Configuration

A bacterial cell that comprises a conjugative plasmid, wherein the cell does not comprise a hypC2 protein or a homologue thereof.

In a third Configuration

Use of a plasmid according to the invention in the manufacture of a composition comprising a first population of cells comprising the plasmid, for enhancing the frequency of plasmid conjugative transfer from the first cells to cells of a second population when first and second cells are in contact with each other.

In a Fourth Configuration

A method of transferring a nucleic acid sequence of interest (NSI) from a donor cell to a recipient cell, wherein the NSI is comprised by a plasmid and the plasmid is comprised by the donor cell, the method comprising combining the cells to allow conjugative tranfer of the plasmid from the donor cell to the recipient cell, wherein the plasmid is according to the invention.

In a Fifth Configuration

A method of treating an infection in a human or animal subject, wherein the infection is an infection of a plurality of bacterial cells (recipient cells), the method comprising

(i) administering a plurality of donor bacterial cells to the subject whereby recipient cells are combined with a donor cells, wherein each donor cell comprises a plasmid according to the invention;

(ii) and allowing conjugative transfer of plasmids from the donor cells to the recipient cells, wherein each transferred plasmid comprises a respective nucleic acid sequence of interest (NSI) that comprises or encodes an antibacterial agent or component thereof that is toxic to recipient cells, whereby recipient cells are killed or the growth or proliferation of recipient cells is inhibited.

In a Sixth Configuration

A plasmid or cell of the invention for use in the method of the fifth configuration.

In a Seventh Configuration

A method of producing a conjugative plasmid that is devoid of a functional hypC2 nucleotide sequence or a homologue thereof, the method comprising
(a) providing a first conjugative plasmid that comprises a first nucleotide sequence which is a hypC2 nucleotide sequence or a homologue thereof that is functional to express a hypC2 protein; and
(b) deleting the first sequence from the first plasmid or mutating the first sequence to render the sequence non-functional for expression of said protein, thereby producing said conjugative plasmid.

In an Eighth Configuration

A conjugative plasmid that is an engineered version of a reference plasmid, wherein the reference plasmid comprises a first nucleotide sequence comprising a hypC2 nucleotide sequence or a homologue thereof that is functional to express a hypC2 protein, wherein the engineering has deleted or rendered non-functional the hypC2 nucleotide sequence or a homologue thereof.

### BRIEF DESCRIPTION OF FIGURES

FIG. 1. The hypc2 KO (knock-out) plasmid mutant shows an approximately 1000× higher conjugation rate compared to the wildtype (WT). Showed is the number of transconjugants (infected cells) obtained from the same amount of donor and incubation time with the WT and optimized (mutant) plasmids.

FIG. 2: A phylogram showing the relatedness of hypC2 found in individual examples of plasmids found in different bacterial host species.

FIG. 3: (a) Shows the presence of hyp2C in multiple incX plasmids (a sequence portion of which is represented by a respective line in the figure). The Hyp2C gene can be located via its location relative to the hypC1 gene (a name we used for commonly-present ORF) (whose gene arrangement is shown schematically in (b) for the pX1 plasmid as well as three other incX plasmids) and close proximity to the stb type toxin-antitoxin genes. Furthermore, it is consistently found upstream the conjugative operon (starting with taxA or homologs hereof) see (a).

### DETAILED DESCRIPTION

The invention is based on the surprising finding that inactivation of a functional hypC2 gene in conjugative plasmids massively enhances the efficiency of conjugation between donor cells harbouring a plasmid and recipient cells to which the plasmid is to be transferred. Inactivation may, for example, be achieved by deleting the gene or part thereof or by inserting one or more exogenous sequences into the gene such that the gene is non-functional for expressing its cognage hypC2 protein. Optionally, a promoter or other regulatory element of the gene may be inactivated (eg, deleted) to prevent gene expression.

Additionally, the inventor has surveyed many different plasmids and has found that this gene and its homologues are widespread amongst conjugative plasmids. The invention, therefore, provides a generally applicable way of enhancing conjugative transfer of plasmids that has many therapeutic and non-therapeutic uses.

Herein, “donor cells” is used interchangeably with “first cells” or “carrier cells”; and “recipient cells” is used interchangeably with “second cells” or “target cells”.

To this end, the invention provides a conjugative plasmid that is devoid of a hypC2 nucleotide sequence or a homologue thereof.

The invention also provides:

A conjugative plasmid that is an engineered version of a reference plasmid, wherein the reference plasmid comprises a first nucleotide sequence comprising a hypC2 nucleotide sequence or a homologue thereof that is functional to express a hypC2 protein, wherein the engineering has deleted or rendered non-functional the hypC2 nucleotide sequence or a homologue thereof.

Optionally, the the reference plasmid is selected from the plasmid of Genbank® accession number HG963477.1, CP023137.2, CP005391.2, CP013972.1, CP017588.1 or CP026699.1; or a homologue of a said selected plasmid, wherein the homologue is capable of being conjugatively transferred (i) to an Enterobacteriaceae cell; or (ii) an E. coli, Klebsiella, Salmonella, Erwinia, Shigella, Pantoea, Proteus or Citrobacter cell.

The invention also provides:

A conjugative plasmid that is obtainable by the method of the seventh configuration or method of producing a plasmid as described below.

The invention provides:

A method of producing a conjugative plasmid that is devoid of a functional hypC2 nucleotide sequence or a homologue thereof, the method comprising
(a) providing a first conjugative plasmid that comprises a first nucleotide sequence which is a hypC2 nucleotide sequence or a homologue thereof that is functional to express a hypC2 protein; and
(b) deleting the first sequence from the first plasmid or mutating the first sequence to render the sequence non-functional for expression of said protein, thereby producing said conjugative plasmid.

Routine molecular biology techniques, such as recombinant DNA technology (eg, recombineering) as will be known by the skilled addressee, can be used to delete or render non-functional the first sequence. For example, the first sequence or part thereof may be deleted, such as by using a DNA vector in homologous recombination with the first plasmid wherein the homologous recombination event deletes the first sequence (or a part), thereby rendering the resulting plasmid non-functional for expression of a hypC2 protein. Additionally or alternatively, one or more nucleic acid sequences may be inserted into and/or adjacent to the first sequence, thereby rendering it non-functional for expressin of hypC2 protein.

Optionally, the first plasmid is selected from the plasmid of Genbank® accession number HG963477.1, CP023137.2, CP005391.2, CP013972.1, CP017588.1 or CP026699.1. Optionally, the first plasmid is an IncX plasmid, eg, a pX1.0 plasmid, pOLA52, pIS15_43, pDSJ07 or R6K plasmid.

Optionally, the conjugative plasmid or plasmid of the invention is a modified plasmid selected from the plasmid of Genbank® accession number HG963477.1, CP023137.2, CP005391.2, CP013972.1, CP017588.1 or CP026699.1.

Optionally, the conjugative plasmid or plasmid of the invention is an engineered version of a reference plasmid, wherein the reference plasmid is selected from the plasmid of Genbank® accession number HG963477.1, CP023137.2, CP005391.2, CP013972.1, CP017588.1 or CP026699.1, wherein the reference plasmid comprises a first nucleotide sequence comprising a hypC2 nucleotide sequence or a homologue thereof that is functional to express a hypC2 protein, wherein the engineering has deleted or rendered non-functional the hypC2 nucleotide sequence or a homologue thereof.

In an example, the first nucleotide sequence comprises SEQ ID NO: 1 or a nucleotide sequence that is at least 70, 80, 85, 90, 95, 96, 97, 98 or 99% identical to SEQ ID NO: 1. In an example, the first nucleotide sequence comprises a sequence selected from SEQ ID NOs: 1 and 3-7 or a nucleotide sequence that is at least 70, 80, 85, 90, 95, 96, 97, 98 or 99% identical to said selected sequence.

In an example, the hypC2 protein comprises SEQ ID NO: 2 or a nucleotide sequence that is at least 70, 80, 85, 90, 95, 96, 97, 98 or 99% identical to SEQ ID NO: 2.

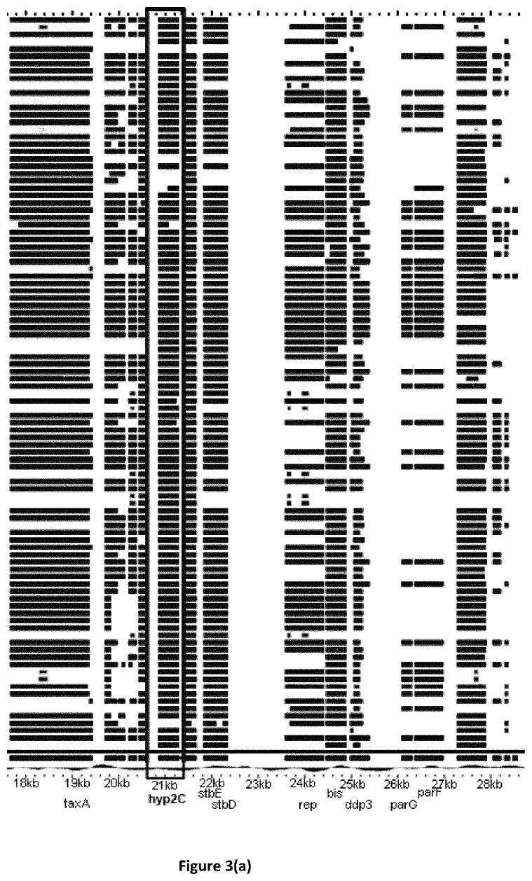

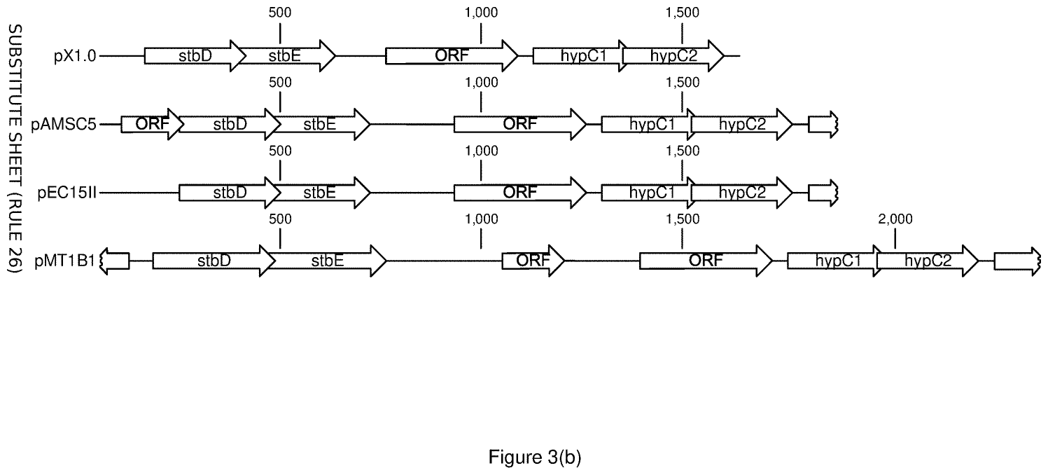

As seen in FIG. 3, hyp2C is located in close proximity to stb type toxin-antitoxin genes (within 1-5 kb of a stb gene). Furthermore, it is consistently found upstream the conjugative operon (starting with taxA or homologs hereof) (within 1-5 kb of a taxA gene).

In an example, the homologue comprises a nucleotide sequence that is at least 75, 76, 77, 78, 79, 80, 85, 90, 95, 96, 97, 98 or 99% identical to SEQ ID NO:1. See Example 2 as an illustration. Additionally or alternatively, the homologue is at a position in the plasmid that corresponds to positions 20849-21064 in plasmid pX1.0.

Optionally, the plasmid is devoid of SEQ ID NO: 1 or a nucleotide sequence that is at least 70% identical to SEQ ID NO: 1. Optionally, the plasmid is devoid of SEQ ID NO: 1 or a nucleotide sequence that is at least 60, 70, 75, 80, 85, 90, 91, 92, 93, 94, 95, 96, 97, 98 or 99% identical to SEQ ID NO: 1.

Preferably, the plasmid is an IncX plasmid, eg, a pX1.0 plasmid.

In an example, the plasmid comprises an OriT of a plasmid selected from an IncX plasmid (eg, a pX1.0 plasmid, pOLA52, pIS15_43, pDSJ07 or R6K plasmid).

In an example, the plasmid is a pIS15_43, pCFSAN002069, pOLA52, R6K or pDSJ07 plasmid, ie, comprises a backbone of such a plasmid. As is known to the skilled addressee, a plasmid backbone comprises an oriV. The backbone may further comprise one or genes required for plasmid replication and/or conjugation.

Optionally, the plasmid is an Enterobacteriaceae plasmid. In an example the plasmid is an E. coli, Klebsiella, Salmonella, Erwinia, Shigella, Pantoea, Proteus or Citrobacter plasmid.

In an example, the plasmid is capable of replicating in an E. coli, Klebsiella, Salmonella, Erwinia, Shigella, Pantoea, Proteus or Citrobacter host cell. In an example, the plasmid is capable of replicating in a cell of a species or genus disclosed in Table 2.

Optionally, the plasmid is capable of being hosted in an Enterobacteriaceae cell.

Optionally, the plasmid is capable of being hosted in an E. coli, Klebsiella, Salmonella, Erwinia, Shigella, Pantoea, Proteus or Citrobacter cell.

Optionally, the plasmid is capable of being conjugatively transferred to an Enterobacteriaceae cell, eg, wherein the cell is of an Enterobacteriaceae species or genus disclosed in Table 2.

In an example, the plasmid is capable of being conjugatively transferred to a cell of a species or genus disclosed in Table 2.

Optionally, the plasmid is capable of being conjugatively transferred to an E. coli, Klebsiella (eg, K. pneumoniae), Salmonella (eg, S. typhimurium), Erwinia, Shigella, Pantoea, Proteus or Citrobacter cell.

The invention also provides a bacterial cell that comprises a conjugative plasmid, wherein the cell does not comprise a hypC2 protein or a homologue thereof.

For example, the cell does not comprise a protein comprising an amino acid sequence that is at least 70, 75, 80, 85, 90, 95, 96, 97, 98 or 99 identical to SEQ ID NO: 2. For example the cell does not comprise a protein comprising SEQ ID NO: 2, eg, wherein the plasmid is an IncX (eg, pX1.0) plasmid. For example, the plasmid comprises an OriT of an IncX (eg, pX1.0) plasmid. For example, the plasmid is capable of being recognized with (ie, operable with) a conjugation system of an IncX plasmid.

The invention also provides a population of such bacterial cells. For example, the population comprises at least 10³, 10⁴, 10⁵, 10⁶, 10⁷, 10⁸, 10⁹, 10¹⁰, 10¹¹, 10¹², 10¹³1 or 0¹⁴ of such cells.

Optionally, each cell comprises at least 2, 3, 4,5, 6, 7, 8, 9, 10, 11, 12, 13, 14, 15, 16, 17, 18, 19, 20, 25, 30, 35, 40, 45, 50, 55, 60, 70, 80, 90 or 100 plasmids of the invention. Optionally, all of the plasmids of the invention comprised by a cell are identical. Alternatively, a cell comprises two, three or more different types of plasmids of the invention. For example, the plasmids may be identical but differ in NSIs.

Optionally, the cell is devoid of SEQ ID NO: 2 or an amino acid sequence that is at least 60, 70, 75, 80, 85, 90, 91, 92, 93, 94, 95, 96, 97, 98 or 99% identical to SEQ ID NO: 2.

Optionally, the cell is an Enterobacteriaceae cell. Optionally, the the cell is an E. coli, Klebsiella, Salmonella, Erwinia, Shigella, Pantoea, Proteus or Citrobacter cell.

In an embodiment, the plasmid comprises or encodes an antibacterial agent, or a component of such an agent. For example, the plasmid encodes an RNA that has antibacterial activity (eg, a silencing RNA). For example, the plasmid encodes a protein that has antibacterial activity or wherein the protein is a component of an antibactertial agent. Thus, once transferred into a recipient cell (or target cell or second cell—both of which are used herein interchangeably with “recipient cell”) the plasmid may express the agent or component inside the cell, wherein the agent kills the cell or reduces the growth or proliferation of the cell (or the component combines in the cell with other component(s) to form such an agent). In an example, the plasmid encodes a guide RNA or crRNA, optionally wherein the guide RNA or crRNA is capable of hybridising to a protospacer sequence of a target cell. Thus, the guide RNA or crRNA guides a Cas in the cell to modify (eg, cut) the protospacer, whereby the cell is killed or its growth or proliferation is inhibited. In an example the Cas is Cas9 or Cas3. The Cas may be a Type I, II, III, IV or V Cas. The Cas may be a nickase or may cut dsDNA. The Cas may be an RNAase.

Optionally, the target cell is selected from an E. coli, Klebsiella (eg, K. pneumoniae), Salmonella (eg, S. typhimurium), Erwinia, Shigella, Pantoea, Proteus or Citrobacter cell. In an example the target cell is a Pseudomonas (eg, P. aeruginosa) cell.

The invention further provides:

Use of plasmids according to the invention in the manufacture of a composition comprising a first population of cells comprising the plasmids, for enhancing the frequency of plasmid conjugative transfer from the first cells (donor cells) to cells (recipient cells) of a second population when first and second cells are in contact with each other and are bacterial cells.

In an example, the recipient cells may be comprised by a surface (eg, on medical equipmenmt or on an apparatus), a gas (eg, air) or a liquid (eg, water or an aqueous liquid, or a petrochemical liquid such as liquid oil or gasolene).

In an example, the frequency is enhanced at least 10, 100 or 1000 times the frequency of a control plasmid that is identical to the plasmid of the invention but further comprises a hypC2 gene that is capable of expressing a hypC2 encoded protein. For example, the gene comprises SEQ ID NO: 1 (or a homologue thereof) and the protein comprises SEQ ID NO: 2 (or a homologue thereof).

Optionally the second cells are comprised by a human or animal subject. Alternatively, the second cells are ex vivo or in vitro. In an example, the second cells are in an environment (ie, not in a human or animal), eg, the second cells are comprised by water, soil, a plant, a field, a waterway, an oil field, oil, a petroleum product, a foodstuff or ingredient thereof, a beverage or an ingredient thereof, air, a gas, or an air or liquid heating or cooling apparatus.

The invention further provides:

A method of transferring a nucleic acid sequence of interest (NSI) from a donor cell to a recipient cell, wherein the NSI is comprised by a plasmid and the plasmid is comprised by the donor cell, the method comprising combining the cells to allow conjugative tranfer of the plasmid from the donor cell to the recipient cell, wherein the plasmid is according to the invention.

In an embodlment, the method is carried out with a plurality of donor and recipient cells, wherein a plurality of donor cells is combined with a plurality of recipient cells and plasmids are transferred into recipient cells.

Examples of NSIs are sequences encoding an antibacterial agent or component thereof (eg, as discussed above), an antibody domain (eg, a VH, VL, VHH or C domain), a therapeutic protein, a fertiliser, an herbicide, a pesticide, a metabolic enzyme, a peptide hormone or a signalling (e.g.

quorum sensing) peptide. Optionally, in the method or use the NSI is or encodes an antibacterial agent, optionally wherein the agent is toxic to the recipient cell. Preferably the agent is not toxic to the donor cell, or is less toxic to the donor cell than the recipient cell.

Optionally, in the method or use the donor cell and/or recipient cell is selected from the group consisting of an E. coli, Klebsiella, Salmonella, Erwinia, Shigella, Pantoea, Proteus or Citrobacter cell. For example, the donor cell is an E. coli cell, eg, a strain selected from Nissle (eg, Nissle 1917), S17, DSM 17252, A0 34/86, Mutaflor™, Symbioflor™ and Colinfant™. Optionally in the method or use the recipient cell is an Enterobacteriaceae cell.

The invention further provides:

A method of treating an infection in a human or animal (e.g. pig, cow, horse, dog, cat, sheep or salmon) subject, wherein the infection is an infection of a plurality of bacterial cells (recipient cells), the method comprising

(i) administering a plurality of donor bacterial cells to the subject whereby recipient cells are combined with a donor cells, wherein each donor cell comprises a plasmid according to the invention; (ii) and allowing conjugative transfer of plasmids from the donor cells to the recipient cells, wherein each transferred plasmid comprises a respective nucleic acid sequence of interest (NSI) that comprises or encodes an antibacterial agent or component thereof that is toxic to recipient cells, whereby recipient cells are killed or the growth or proliferation of recipient cells is inhibited.

In one embodiment, the plasmids are identical. In another embodiment different plasmids are used, eg, differing in their NSIs.

In an example, the administration is oral administration. In an example, the administration is intravenous administration. In an example, the administration is administration through a catheter.

In an example, the recipient cells are pathogenic E. coli cells, eg, E. coli ETEC, EPEC, EIEC, EHEC, EAEC or AIEC cells.

The invention provides:

A plasmid or cell of the invention for use in a method according to the invention.

Optionally, in the method or use the donor cell is an E. coli cell (eg, Nissle strain) and the recipient cell (eg, a cell that is pathogenic to humans or animals) is an E. coli, Klebsiella (eg, K. pneumoniae), Salmonella (eg, S. typhimurium), Erwinia, Shigella, Pantoea, Proteus oraz Citrobacter cell.

In an example, the target, second or recipient cell is a cell of a species or strain of bacteria that is pathogenic to humans or animals

In some embodiments, the invention relates to carrier bacteria encoding desired protein or RNA (eg, encoding an antimicrobial agent) and methods of use. In embodiments, the agent can be transferred into target cells by conjugation between carrier cells (to which the agent is not toxic) and the target cells, whereby the agent is toxic to the target cells and kills the target cells. In other embodiments, the growth or proliferation of target cells is reduced (eg, by at least 40, 50, 60, 70, 80, or 90% compared to growth in the absence of the agent). Each carrier cell comprises episomal (ie, plasmid) DNA encoding an antibacterial agent that is toxic to a target bacterial cell but is not toxic (or is less toxic) to the carrier cell. The invention finds application, for example, in controlling or killing target bacteria that are pathogenic to humans, animals or plants. The invention finds application, for example, in controlling or killing zoonotic target bacteria comprised by an animal (eg, a livestock animal) For example, the carrier cells may be comprised by a medicament for treating or preventing a disease or condition in a human or animal; a growth promoting agent for administration to animals for promoting growth theref; killing zoonositic bacteria in the animals; for administration to livestock as a pesticide; a pesticide to be applied to plants; or a plant fertilizer.

An advantage of the invention is that the carrier cells may be used as producer cells in which plasmid DNA encoding the antibacterial agent can be replicated. Additionally or alternatively, the plasmid and the conjugation system may also be separated, such that the cell carries the conjugation system (lacking hyp2C) on its chromosome to mobilize a (any) plasmid containing a compatible oriT.

In certain embodiments, the invention uses sequence-specific killing of the target cell to achieve selectivity. To this end, in an example the plasmid encodes a guided nuclease that is operable in the target cell to recognize and cut a target sequence of a target cell chromosome, thereby killing the cell or wherein the growth or proliferation of the cell is reduced. This is advantageous over the use of other types of toxic agent, which are less discriminate in their action, being able to kill several species or strain (eg, potentially also being toxic to the carrier cell to some degree). By using a guided nuclease (eg, a TALEN or Cas nuclease), these can be programmed to recognize a target sequence that is present in the target cell genome (eg, comprised by a chromosome or episome of the target cell), but is absent in the genome of the carrier cell.

Thus, in this case replication of the plasmid DNA can freely happen in the carrier cell without risk of killing the cell or reducing its growth or proliferation due to the encoded agent and replication of sequences encoding the agent. Thus, in an example, where the plasmid DNA encodes a guided nuclease, the guided nuclease is capable of recognizing and cutting a target nucleic acid sequence comprised by the genome (eg, chromosome) of the target cell, wherein the target cell is absent in the carrier cell.

A particularly useful example is where the plasmid DNA encodes a Cas nuclease (eg, a Cas9 or Cas3) that is operable with a guide RNA or crRNA in the target cell, wherein the RNA is operable to guide the Cas to the target sequence, wherein the Cas modifies (eg, cuts) the target sequence and the target cell is killed or target cell growth or proliferation is inhibited. In one embodiment, the plasmid DNA encodes the Cas and the guide RNA or crRNA. In another embodiment, the plasmid DNA encodes the guide RNA or crRNA, but does not encode a cognate Cas. In this embodiment, the RNA is operable in the target cell with an endogenous Cas encoded by the target cell genome, wherein the RNA is operable to guide the Cas to the target sequence, wherein the Cas modifies (eg, cuts) the target sequence and the target cell is killed or target cell growth or proliferation is inhibited. In this se sense, the agent may comprise a component of a CRISPR/Cas system (eg, a Cas nuclease, Cascade Cas, crRNA, guide RNA or tracrRNA). Thus, the invention usefully recognizes the benefit of using antibacterial agents that act by target recognition in the target cell but not in the carrier cell, which opens up the ability for the plasmid DNA to freely replicated in the carrier cell without significant toxicity to the carrier cell.

In an example, the antibacterial agent comprises a guided nuclease that is capable of recognizing and cutting a target nucleic acid sequence comprised by the target cell genome, wherein the target sequence is not comprised by the carrier cell. In this sense, therefore, the antibacterial agent is toxic to a target bacterial cell but is not toxic to the carrier cell. For example, the agent is a component of a CRISPR/Cas system that is operable in the target cell to modify a target nucleic acid sequence comprised by the target cell genome (eg, comprised by the target cell chromosome).

Optionally, the nuclease is a Cas nuclease, meganuclease, zinc finger nuclease or TALEN. Optionally, the nuclease is a Cas nuclease of a Type I, II, III, IV or V CRISPR system. In an aspect, the component of the antibacterial agent is a guide RNA or crRNA that is capable of hybridising to the target sequence of the target cell. They system may be a Type I, II, III, IV or V CRISPR system.

The protospacer sequence be comprised by a chromosome or episome (eg, plasmid) of the carrier cell.

Advantageously, the donor cell(s) or plasmid(s) is (are) for treating or preventing a target cell infection in a human or an animal subject (eg, a chicken, cow, pig, fish or shellfish). Advantageously, the carrier cell is a cell of a species that is probiotic to said subject or is probioitic to humans or animals (eg, chickens). For example, the carrier cell is a probiotic E. coli cell. In an example, the target cell is a cell of a species that is pathogenic to said subject, or is pathogenic to humans or animals (eg, chickens). Advantageously, the plasmid DNA encodes one or more guide RNAs or one or more crRNAs that are capable of hybridizing in the target cell to respective target nucleic acid sequence(s), wherein the target sequence(s) are comprised by an endogenous chromosome and/or endogenous episome of the target cell. For example, the plasmid DNA encodes 2, 3, 4, 5, 6, 7, 7, 9, or 10 (or more than 10) different gRNAs or different crRNAs that hybridise to a respective target sequence, wherein the target sequences are different from each other. For example, 3 different gRNAs or crRNAs are encoded by the plasmid DNA. For example, 2 different gRNAs or crRNAs are encoded by the plasmid DNA. For example, 3 different gRNAs or crRNAs are encoded by the plasmid DNA. For example, 4 different gRNAs or crRNAs are encoded by the plasmid DNA. For example, 3 different gRNAs or crRNAs are encoded by the plasmid DNA. For example, 5 different gRNAs or crRNAs are encoded by the plasmid DNA. For example, 6 different gRNAs or crRNAs are encoded by the plasmid DNA. For example, 7 different gRNAs or crRNAs are encoded by the plasmid DNA. For example, 8 different gRNAs or crRNAs are encoded by the plasmid DNA. For example, 9 different gRNAs or crRNAs are encoded by the plasmid DNA. For example, 10 different gRNAs or crRNAs are encoded by the plasmid DNA. For example, 11 different gRNAs or crRNAs are encoded by the plasmid DNA. For example, 12 different gRNAs or crRNAs are encoded by the plasmid DNA. For example, 13 different gRNAs or crRNAs are encoded by the plasmid DNA. In an example, the target cells are Salmonella cells (eg, wherein the subject is a chicken). In an example, the target cells are E. coli, Pseudomonas, Klebsiella or C. dificile cells. In an example, the target cells are Campylobacter cells (eg, wherein the subject is a chicken). In an example, the target cells are Edwardsiella cells (eg, wherein the subject is a fish or shellfish, eg, a catfish or a shrimp or prawn). In an example, the target cells are E. coli cells.

In an alternative herein, the carrier and target cells are archaeal cells.

In a preferred example, the NSI encodes an antibacterial agent that is toxic to a target bacterial cell but is not toxic to the carrier cell. In an example, the NSI encodes an antibiotic agent, an antibody, an antibody chain or an antibody variable domain. In an example, the NSI encodes a guide RNA or a crRNA that is operable in the target cell with a cognate Cas (eg, a Cas nuclease to target and cut a protospacer sequence comprised by a chromosome or episome of the target cell). In an example the RNA is a siRNA that is capable of hybridizing to an endogenous target nucleic acid sequence of the target cell to silence transcription and/or translation thereof.

In an example, the plasmid comprises an expressible tra1 and/or tra2 module or a homologue thereof.

Optionally, the carrier cell is an E. coli (eg, Nissle, or S17 E. coli strain) or Lactobacillus cell or Bacillus cell or Enterococcus cell. Optionally, the carrier cell is a cell of a human, chicken pig, sheep, cow, fish (eg, catfish or salmon) or shellfish (eg, shrimp or lobster) commensal bacterial strain (eg, a commensal E. coli strain).

Optionally, the carrier cell or plasmid is for administration to a microbiota of a human or animal subject for medical use. For example, the medical use is for treating or preventing a disease disclosed herein. For example, the medical use is for treating or preventing a condition disclosed herein. For example, the medical use is for the treatment or prevention of a disease or condition mediated by said target cells. For example, the carrier cell or plasmid for administration to an animal for enhancing growth or weight of the animal

In an example, the administration in the method of the invention is to a human for enhancing the growth or weight of the human Optionally, the enhancing is not a medical therapy. Optionally, the enhancing is a medical therapy. Optionally, the method comprises the administration of a plurality of carrier cells to a microbiota (eg, a gut microbiota) of the subject, wherein the microbiota comprises target cells and plasmid DNA is transferred into target cells for expression therein to produce the antibacterial agent, thereby killing target cells in the subject or reducing the growth or proliferation of target cells.

Optionally, the use or method may be carried out to target cells (recipient cells) comprised by a plant, eg, to fertilise the plant or as a herbicide treatment. The plant may be any plant disclosed herein. For example a plant herein in any configuration or embodiment of the invention is selected from a tomato plant, a potato plant, a wheat plant, a corn plant, a maize plant, an apple tree, a bean-producing plant, a pea plant, a beetroot plant, a stone fruit plant, a barley plant, a hop plant and a grass. For example, the plant is a tree, eg, palm, a horse chestnut tree, a pine tree, an oak tree or a hardwood tree. For example the plant is a plant that produces fruit selected from strawberries, raspberries, blackberries, reducrrants, kiwi fruit, bananas, apples, apricots, avoocados, cherries, oranges, clementines, satsumas, grapefruits, plus, dates, figs, limes, lemons, melons, mangos, pears, olives or grapes. Optionally, the plant is a dicotyledon. Optionally, the plant is a flowering plant. Optionally, the plant is a monocotyledon.

In any configuration (eg, wherein applied to a plant), embodiment or example herein, the target bacteria are P. syringae bacteria (eg, comprised by a plant). Pseudomonas syringae pv. syringae is a common plant-associated bacterium that causes diseases of both monocot and dicot plants worldwide. In an example the targt bacteria are P. syringae bacteria of a pathovar selected from P. s. pv. aceris, P. s. pv. aptata, P. s. pv. atrofaciens, P. s. pv. dysoxylis, P. s. pv. japonica, P. s. pv. lapsa, P. s. pv. panici, P. s. pv. papulans, P. s. pv. pisi, P. s. pv. syringae and P. s. pv. morsprunorum.
P. s. pv. aceris attacks maple Acer species.
P. s. pv. actinidiae attacks kiwifruit Actinidia deliciosa.
P. s. pv. aesculi attacks horse chestnut Aesculus hippocastanum, causing bleeding canker.
P. s. pv. aptata attacks beets Beta vulgaris.
P. s. pv. atrofaciens attacks wheat Triticum aestivum.
P. s. pv. dysoxylis attacks the kohekohe tree Dysoxylum spectabile.
P. s. pv. japonica attacks barley Hordeum vulgare.
P. s. pv. lapsa attacks wheat Triticum aestivum.
P. s. pv. panici attacks Panicum grass species.
P. s. pv. papulans attacks crabapple Malus sylvestris species.
P. s. pv. phaseolicola causes halo blight of beans.
P. s. pv. pisi attacks peas Pisum sativum.
P. s. pv. syringae attacks Syringa, Prunus, and Phaseolus species.
P. s. pv. glycinea attacks soybean, causing bacterial blight of soybean.

In an example, the target bacteria are P. syringae selected from a serovar recited in a bullet point in the immediately preceding paragraph and the bacteria are comprised by a plant also mentioned in that bullet point.

When the method or use is applied to a plant, the carrier cells may be combined with a microbiota comprising the recipient cells. The microbiota is comprised by a leaf, trunk, root or stem of the plant. In an example, the target bacteria (or taraget cell) is comprised by a microbiota of a plant. In an example, the microbiota is comprised by a leaf. In an example, the microbiota is comprised by a xylem. In an example, the microbiota is comprised by a phloem. In an example, the microbiota is comprised by a root. In an example, the microbiota is comprised by a tuber. In an example, the microbiota is comprised by a bulb. In an example, the microbiota is comprised by a seed. In an example, the microbiota is comprised by an exocarp, epicarp, mesocarp or endocarp. In an example, the microbiota is comprised by a fruit, eg, a simple fruits; aggregate fruits; or multiple fruits. In an example, the microbiota is comprised by a seed or embryo, eg, by a seed coat; a seed leaf; cotyledons; or a radicle. In an example, the microbiota is comprised by a flower, eg, comprised by a peduncle; sepal: petals; stamen; filament; anther or pistil. In an example, the microbiota is comprised by a root; eg, a tap root system, or a fibrous root system. In an example, the microbiota is comprised by a leaf or leaves, eg, comprised by a leaf blade, petiole or stipule. In an example, the microbiota is comprised by a stem, eg, comprised by bark, epidermis, phloem, cambium, xylem or pith.

The invention provides:

A method for reducing a biofilm comprised by a subject or comprised on a surface, wherein the biofilm comprises target cells (eg, Pseudomonas cells), wherein the method comprises the administration of a plurality of carrier cells according to the invention to the biofilm, wherein plasmid DNA is transferred from carrier cells into target cells for expression therein to produce the antibacterial agent, thereby killing target cells in the biofilm or reducing the growth or proliferation of target cells.

In an example “reducing a biofilm” comprises reducing the coverage area of the biofilm. In an example “reducing a biofilm” comprises reducing the proliferation of the biofilm. In an example “reducing a biofilm” comprises reducing the durability of the biofilm. In an example “reducing a biofilm” comprises reducing the spread of the biofilm (eg, in or on the subject, eg, spread to the environment containing the subject).

In an example, the subject is a human or animal For example, the biofilm is comprised by a lung of the subject, eg, wherein the target cells are Pseudomonas (eg, P aeruginosa) cells. This may be useful wherein the subject is a human suffering from a lung disease or condition, such as pneumonia or cystic fibrosis. For example, the biofilm is comprised by an animal or human organ. For example, the biofilm is comprised by a microbiota of a human or animal.

In an example, the target bacteria (or taraget cell) is comprised by a biofilm of a plant. In an example, the biofilm is comprised by a leaf. In an example, the biofilm is comprised by a xylem. In an example, the biofilm is comprised by a phloem. In an example, the biofilm is comprised by a root. In an example, the biofilm is comprised by a tuber. In an example, the biofilm is comprised by a bulb. In an example, the biofilm is comprised by a seed. In an example, the biofilm is comprised by an exocarp, epicarp, mesocarp or endocarp. In an example, the biofilm is comprised by a fruit, eg, a simple fruits; aggregate fruits; or multiple fruits. In an example, the biofilm is comprised by a seed or embryo, eg, by a seed coat; a seed leaf; cotyledons; or a radicle. In an example, the biofilm is comprised by a flower, eg, comprised by a peduncle; sepal: petals; stamen; filament; anther or pistil. In an example, the biofilm is comprised by a root; eg, a tap root system, or a fibrous root system. In an example, the biofilm is comprised by a leaf or leaves, eg, comprised by a leaf blade, petiole or stipule. In an example, the biofilm is comprised by a stem, eg, comprised by bark, epidermis, phloem, cambium, xylem or pith.

Optionally, the surface is a surface ex vivo, such as a surface comprised by a domestic or industrial apparatus or container.

Optionally, the target cells are comprised by a biofilm, eg, a biofilm as disclosed herein.

Optionally, the target bacteria are Salmonella, Pseudomonas, Escherichia, Klebsiella, Campylobacter, Helicobacter, Acinetobacter, Enterobacteriacea, Clostridium, Staphylococcus or Streptococcus bacteria. For example, the target bacteria are Salmonella enterica bacteria. For example, the target bacteria are selected from the group consisting of Salmonella enterica subsp. enterica, serovars Typhimurium, Enteritidis, Virchow, Montevideo, Hadar and Binza. For example, the target bacteria are Pseudomonas (eg, P. syringae or P. aeruginosa) bacteria.

In an embodiment, the target bacteria are E. coli bacteria. Optionally, the target bacteria are enterohemorrhagic E. coli (EHEC), E. coli Serotype 0157:H7 or Shiga-toxin producing E. coli (STEC)). In an example, the taraget bacteria are selected from
Shiga toxin-producing E. coli (STEC) (STEC may also be referred to as Verocytotoxin-producing E. coli (VTEC);
Enterohemorrhagic E. coli (EHEC) (this pathotype is the one most commonly heard about in the news in association with foodborne outbreaks);
Enterotoxigenic E. coli (ETEC);
Enteropathogenic E. coli (EPEC);
Enteroaggregative E. coli (EAEC);
Enteroinvasive E. coli (EIEC); and
Diffusely adherent E. coli (DAEC).
   Enterohemorrhagic Escherichia coli (EHEC) serotype O157:H7 is a human pathogen responsible for outbreaks of bloody diarrhoea and haemolytic uremic syndrome (HUS) worldwide. Conventional antimicrobials trigger an SOS response in EHEC that promotes the release of the potent Shiga toxin that is responsible for much of the morbidity and mortality associated with EHEC infection. Cattle are a natural reservoir of EHEC, and approximately 75% of EHEC outbreaks are linked to the consumption of contaminated bovine-derived products. EHEC causes disease in humans but is asymptomatic in adult ruminants Characteristics of E. coli serotype O157:H7 (EHEC) infection includes abdominal cramps and bloody diarrhoea, as well as the life-threatening complication haemolytic uremic syndrome (HUS). Currently there is a need for a treatment for EHEC infections (Goldwater and Bettelheim, 2012). The use of conventional antibiotics exacerbates Shiga toxin-mediated cytotoxicity. In an epidemiology study conducted by the Centers for Disease Control and Prevention, patients treated with antibiotics for EHEC enteritis had a higher risk of developing HUS (Slutsker et al., 1998). Additional studies support the contraindication of antibiotics in EHEC infection; children on antibiotic therapy for hemorrhagic colitis associated with EHEC had an increased chance of developing HUS (Wong et al., 2000; Zimmerhackl, 2000; Safdar et al., 2002; Tan et al., 2005). Conventional antibiotics promote Shiga toxin production by enhancing the replication and expression of stx genes that are encoded within a chromosomally integrated lambdoid prophage genome. The approach of some configurations of present invention rely on nuclease cutting. Stx induction also promotes phage-mediated lysis of the EHEC cell envelope, allowing for the release and dissemination of Shiga toxin into the environment (Karch et al., 1999; Matsushiro et al., 1999; Wagner et al., 2002). Thus, advantageously, these configurations of the invention provide alternative means for treating EHEC in human and animal subjects. This is exemplified below with surprising results on the speed and duration of anti-EHEC action produced by nuclease action (as opposed to conventional antibiotic action).

In an example, the subject (eg, a human or animal) is suffering from or at risk of haemolytic uremic syndrome (HUS), eg, the subject is suffering from an E. coli infection, such as an EHEC E. coli infection.

The invention provides:

A pharmaceutical composition, livestock growth promoting composition, soil improver, herbicide, plant fertilizer, food or food ingredient sterilizing composition, dental composition, personal hygiene composition or disinfectant composition (eg, for domestic or industrial use) comprising a plurality of carrier cells according to the invention.

Herein, a carrier cell is, eg, a probiotic cell for administration to a human or animal subject. For example, the carrier cell is commensal in a microbiome (eg, gut or blood microbiome) of a human or animal subject, wherein the carrier is for administration to the subject. In an example, a carrier cell is a bacterial cell (and optionally the target cell is a bacterial cell). In an example, a carrier cell is an archaeal cell (and optionally the target cell is an archaeal cell)

Optionally, the carrier cell is a gram-positive bacterial cell and the target cell is a gram-positive bacterial cell.

Optionally, the carrier cell is a gram-positive bacterial cell and the target cell is a gram-negative bacterial cell.

Optionally, the carrier cell is a gram-negative bacterial cell and the target cell is a gram-positive bacterial cell.

Optionally, the carrier cell is a gram-negative bacterial cell and the target cell is a gram-negative bacterial cell.

Optionally, the carrier cell is an E. coli bacterial cell and the target cell is a Pseudomonas bacterial cell.

Optionally, the carrier cell is an E. coli bacterial cell and the target cell is a gram-positive bacterial cell.

Optionally, the carrier cell is an E. coli bacterial cell and the target cell is a gram-netative bacterial cell.

Optionally, the carrier cell is an E. coli bacterial cell and the target cell is a Salmonella bacterial cell.

Optionally, the carrier cell is an E. coli bacterial cell and the target cell is an E. coli bacterial cell.

Optionally, the carrier cell is an E. coli bacterial cell and the target cell is a Pseudomonas bacterial cell.

Optionally, the carrier cell is a probiotic or commensal E. coli bacterial cell for administration to a human or animal subject.

Herein, optionally the plasmid comprises a closed circular DNA. In an embodiment, in an example the plasmid DNA is dsDNA. In an embodiment, in an example the plasmid DNA is ssDNA.

Optionally, the target cell is a Salmonella cell (eg, wherein the carrier cell is an E. coli cell), eg, a Salmonella enterica subsp. enterica, eg, a Salmonella enterica subsp. enterica serovar Typhimurium, Enteritidis, Virchow, Montevideo, Hadar or Binza.

For example, the target bacteria are selected from the group consisting of S. enterica; S. typhimurium; P. aeruginosa; E. coli; K. pneumoniae; C. jujeni; H. pylori; A. baumanii; C. difficile; S. aureus; S. pyogenes or S. thermophilus.

In an example, the target cell is a cell of a species that causes nosocomial infection in humans.

Optionally, the target cell is comprised by an animal (eg, poultry animal (such as chicken), swine, cow, fish (eg, catfish or salmon) or shellfish (eg, prawn or lobster)) microbiome. Optionally, the microbiome is a gut microbiome. For example, the target cell is a Salmonella cell comprised by a chicken gut biofilm. For example, the target cell is a Salmonella cell comprised by a chicken gut biofilm sample ex vivo.

Optionally, the plurality of carrier cells comprises a first sub-population of carrier cells (first cells) and a second sub-population of carrier cells (second cells) wherein the first cells comprise indentical plasmid DNAs and the second cells comprise indentical plasmid DNAs (which are different from the plasmid DNAs of the first cells). For example, the former DNAs comprise a NSI that is different from the NSI comprised by the other DNAs. For example, the plasmid DNAs encode a first guide RNA or crRNA and the second DNAs encode a second guide RNA or crRNA, wherein the first guide RNA/crRNA is capable of hybridizing to a first protospacer sequence in first target cells; and the second guide RNA/crRNA is capable of hybridizing to a second protospacer sequence in second target cells, wherein the protospacers are different. Optionally, the first target cells are different from the second target cells. Optionally, the first target cells are of the same species or strain as the second target cells. Alternatively, the first target cells are of species or strain that is different from the species or strain of the second target cells (in this way a cocktail of carrier cells is provided, eg, for administration to a human or animal or plant, to target and kill a plurality of target cells of different species or strains).

In an example, the or each plasmid DNA comprises a plurality (eg, a first and a second) of NSIs wherein a first NSI is different from a second NSI (eg, they encode different proteins or RNAs, such as different guide RNAs or crRNAs). In an example, the or each plasmid DNA comprises 2, 3, 4, 5, 6, 7, 8, 9,10, 11, 12, 13, 14, 15, 16, 17, 18, 19 or 20 different types of NSIs. In an example, the or each plasmid DNA comprises NSIs encoding 2, 3, 4, 5, 6, 7, 8, 9,10, 11, 12, 13, 14, 15, 16, 17, 18, 19 or 20 different guide RNAs. In an example, the or each plasmid DNA comprises NSIs encoding 2, 3, 4, 5, 6, 7, 8, 9,10, 11, 12, 13, 14, 15, 16, 17, 18, 19 or 20 different crRNAs. In an example, the or each plasmid DNA comprises NSIs encoding at least 2, 3, 4, 5, 6, 7, 8, 9,10, 11, 12, 13, 14, 15, 16, 17, 18, 19 or 20 different guide RNAs. In an example, the or each plasmid DNA comprises NSIs encoding at least 2, 3, 4, 5, 6, 7, 8, 9,10, 11, 12, 13, 14, 15, 16, 17, 18, 19 or 20 different crRNAs.

Optionally, the composition is comprised by a liquid (eg, an aqueous liquid or in water), the composition comprising the carrier cells at an amount of from 1×10³ to 1×10¹⁰ (eg, from 1×10⁴ to 1×10¹⁰; from 1×10⁴ to 1×10⁹; from 1×10⁴to 1×10⁸; from 1×10⁴ to 1×10⁷; from 1×10³ to 1×10¹⁰; from 1×10³ to 1×10⁹; from 1×10³ to 1×10⁸; from 1×10³ to 1×10⁷; from 1×10⁵ to 1×10¹⁰; from 1×10⁵ to 1×10⁹; from 1×10⁵ to 1×10⁸; from 1×10⁵ to 1×10⁷; from 1×10⁶ to 1×10¹⁰; from 1×10⁶ to 1×10⁹; from 1×10⁶ to 1×10⁸; or from 1×10⁶ to 1×10⁷) cfu/ml. For example, the liquid is a beverage, such for human or animal consumption. For example, the beverage is a livestock beverage, eg, a poultry beverage (ie, a beverage for consumption by poultry, such as chicken).

In an example, the composition is a dietary (eg, dietary supplement) composition for consumption by humans or animals In an example, the composition is a slimming composition for consumption by humans or animals In an example, the composition is a growth promotion composition for consumption by humans or animals In an example, the composition is a body buidling composition for consumption by humans. In an example, the composition is a probiotic composition for consumption by humans or animals In an example, the composition is a biocidal composition for consumption by humans or animals In an example, the composition is a pesticidal composition for consumption by humans or animals In an example, the composition is a zoonosis control composition for consumption by animals

In an example, the composition comprises vitamins in addition to the carrier cells. In an example, the composition comprises vitamin A, B (eg, B12), C, D, E and/or K in addition to the carrier cells. In an example, the composition comprises lipids in addition to the carrier cells. In an example, the composition comprises carbohydrates in addition to the carrier cells. In an example, the composition comprises proteins and/or amino acids in addition to the carrier cells. In an example, the composition comprises minerals in addition to the carrier cells. In an example, the composition comprises metal ions (eg, Mg²⁺, Cu²⁺ and/or Zn²⁺) in addition to the carrier cells. In an example, the composition comprises sodium ions, potassium ions, magnesium ions, calcium ions, manganese ions, iron ions, cobalt ions, copper ions, zinc ions and/or molybdenum ions.

In an example, the composition is a plant fertilizer composition. In an example, the composition is a herbicide. In an example, the composition is a pesticide composition for application to plants.

In any embodiment or example, where appropriate: The plants are, for example, crop plants. The plants are, for example, wheat. The plants are, for example, corn. The plants are, for example, maize The plants are, for example, fruiting plants. The plants are, for example, vegetable plants. The plants are, for example, tomato plants. The plants are, for example, potato plants. The plants are, for example, grass plants. The plants are, for example, flowering plants. The plants are, for example, trees. The plants are, for example, shrubs.

In an example, the composition is for environmental application, wherein the environment is an outdoors environment (eg, application to a field or waterway or reservoir).

In an example, the composition is comprised by a food or food ingredient (eg, for human or animal consumption). In an example, the composition is comprised by a beverage or beverage ingredient (eg, for human or animal consumption).

In an example the target cell(s) are human biofilm cells, eg, wherein the biofilm is a gut, skin, lung, eye, nose, ear, gastrointestinal tract (GI tract), stomach, hair, kidney, urethra, bronchiole, oral cavity, mouth, liver, heart, anus, rectum, bladder, bowel, intestine, penis, vagina or scrotum biofilm. In an example the target cell(s) are animal biofilm cells, eg, wherein the biofilm is a gut, skin, lung, eye, nose, ear, gastrointestinal tract (GI tract), caecum, stomach, hair, feather, scales, kidney, urethra, bronchiole, oral cavity, mouth, liver, heart, anus, rectum, bladder, bowel, intestine, penis, vagina or scrotum biofilm. For example, the biofilm is a bird (eg, chicken) caecum biofilm.

In an example, any method herein is ex vivo. In an example, a method herein is in vivo. In an example, a method herein is in vitro. In an example, a method herein is carried out in an environment, eg, in a domestic (such as in a house), industrial (such as in a factory) or agricultural environment (such as in a field). In an example, a method herein is carried out in or on a container; or on a surface.

In an example, the NSI (or a RNA product thereof) is capable of recombination with the target cell chromosome or an episome comprised by the target cell to modify the chromosome or episome. Optionally, this is carried out in a method wherein the chromosome or episome is cut (eg, at a predetermined site using a guided nuclease, such as a Cas, TALEN, zinc finger nuclease or meganuclease) and simultaneously or sequentially the plasmid DNA is introduced into the target cell by conjugation with the carrier cell and the NSI or a sequence thereof is inserted into the chromosome or episome at or adjacent the cut site.

In an example the plasmid DNA comprises one or more components of a CRISPR/Cas system operable to perform protospacer cutting in the target cell (eg, wherein the protospacer comprises 10-20, 10-30, 10-40, 10-100, 12-15 or 12-20 consecutive nucleotides that are capable of hybridizing in the target cell with a crRNA or gRNA encoded by the NSI).

For example, the system is a Type I, II, III, IV or V CRISPR/Cas system.

In an example, the NSI encodes a Cas9 (and optionally a second, different, Cas, such as a Cas3, Cas9, Cpf1, Cas13a, Cas13b or Cas10). In an example, the NSI encodes a Cas3 (and optionally a second, different, Cas, such as a Cas3, Cas9, Cpf1, Cas13a, Cas13b or Cas10). In an example, the NSI encodes a Cas selected from a Cas3, Cas9, Cpf1, Cas13a, Cas13b and Cas10. Additionally or alternatively, the plasmid DNA (eg, the NSI) encodes a guide RNA or crRNA or tracrRNA. For example, the guide RNA or crRNA or tracrRNA is cognate to (ie, operable with in the target cell) the first Cas.

In an example, a Cas herein is a Cas9. In an example, a Cas herein is a Cas3. The Cas may be identical to a Cas encoded by the target bacteria.

In an embodiment, the presence in the target bacterium of the NSI or its encoded protein or RNA mediates target cell killing, or downregulation of growth or propagation of target cells. In an embodiment, the presence in the target bacterium of the NSI or its encoded protein or RNA mediates switching off of expression of one or more RNA or proteins encoded by the target cell genome, or downregulation thereof.

In an embodiment, the presence in the target bacterium of the NSI or its encoded protein or RNA mediates upregulation of growth or propagation of the target cell. In an embodiment, the presence in the target bacterium of the NSI or its encoded protein or RNA mediates switching on of expression of one or more RNA or proteins encoded by the target cell genome, or upregulation thereof.

In an embodiment, the NSI encodes a component of a CRISPR/Cas system that is toxic to the target bacterium.

In an embodiment, the plasmid is a shuttle vector.

Optionally, the target cell is devoid of a functional endogenous CRISPR/Cas system before transfer therein of the plasmid DNA, eg, a plasmid DNA comprising component of an exogenous CRISPR/Cas system that is functional in the target cell and toxic to the target cell. An embodiment provides an antibacterial composition comprising a plurality of carrier cells of the invention, wherein each target cell is optionally according to this paragraph, for administration to a human or animal subject for medical use.

In an example, the composition of the invention is a herbicide, pesticide, insecticide, plant fertilizer or cleaning agent.

Optionally, target bacteria herein are comprised by a microbiome of the subject, eg, a gut microbiome. Altertnatively, the microbiome is a skin, scalp, hair, eye, ear, oral, throat, lung, blood, rectal, anal, vaginal, scrotal, penile, nasal or tongue microbiome.

In an example the subject (eg, human or animal) is further administered a medicament simultaneously or sequentially with the carrier cell administration. In an example, the medicament is an antibiotic, antibody, immune checkpoint inhibitor (eg, an anti-PD-1, anti-PD-L1 or anti-CTLA4 antibody), adoptive cell therapy (eg, CAR-T therapy) or a vaccine.

In an embodiment, the NSI encodes a guided nuclease, such as a Cas nuclease, TALEN, zinc finger nuclease or meganuclease. Thus, the toxic agent may comprise a guided nuclease, such as a Cas nuclease, TALEN, zinc finger nuclease or meganuclease. Optionally, the NSI encodes a restriction nuclease that is capable of cutting the chromosome of the target cell.

Optionally, the composition is a pharmaceutical composition for use in medicine practised on a human or animal subject.

In an example, the animal is a livestock or companion pet animal (eg, a cow, pig, goat, sheep, horse, dog, cat or rabbit). In an example, the animal is an insect (an insect at any stage of its lifecycle, eg, egg, larva or pupa). In an example, the animal is a protozoan. In an example, the animal is a cephalopod.

Optionally, the composition is a herbicide, pesticide, food or beverage processing agent, food or beverage additive, petrochemical or fuel processing agent, water purifying agent, cosmetic additive, detergent additive or environmental (eg, soil) additive or cleaning agent.

The invention also provides:

A target bacterial cell or a plurality of target bacterial cells each comprising a said plasmid DNA.

For example the carrier bacteria are Lactobacillus (eg, L. reuteri or L. lactis), E. coli or Streptococcus (eg, S. thermophiles) bacteria. Usefully, the carrier can provide protection for the plasmid DNA from the surrounding environment. The use of a carrier may be useful for oral administration or other routes where the carrier can provide protection for the plasmid DNA from the acid stomach or other harsh environments in the subject. Furthermore, the carrier can be formulated into a beverage, for example, a probiotic drink, eg, an adapted Yakult (trademark), Actimel (trademark), Kevita (trademark), Activia (trademark), Jarrow (trademark) or similar drink for human consumption.

Optionally, the carrier cell(s) or composition are for administration to a human or animal subject for medical use, comprising killing target bacteria using the agent or expression product of the NSI, wherein the target bacteria mediate as disease or condition in the subject. In an example, when the subject is a human, the subject is not an embryo. In an example, the carrier cells are probiotic in the subject.

The invention also provides:

A method of killing target bacterial cells in an environment, optionally wherein the method is not practised on a human or animal body, wherein the method comprises exposing the environment to the carrier cell(s) or composition of the invention and allowing the product of the NSI to be expressed in the target cells, wherein the target bacteria are killed in the presence of said product. For example, the product encodes a CRISPR/Cas system or component thereof, such as a system or component disclosed herein. Thus, the system may be capable of recognisisng and cutting a chromosomal protopspacer sequence of the target cell, whereby the target cell is killed. Optionally, in a further step killed target cells are isolated.

The invention also provides:

Use of the composition or cell(s) of the invention, in the manufacture of an antibacterial agent that kills target bacteria, for the treatement of a disease or condition in a human or animal subject comprising the target bacteria.

Optionally, the environment is a microbiome of soil; a plant, part of a part (e.g., a leaf, fruit, vegetable or flower) or plant product (e.g., pulp); water; a waterway; a fluid; a foodstuff or ingredient thereof; a beverage or ingredient thereof; a medical device; a cosmetic; a detergent; blood; a bodily fluid; a medical apparatus; an industrial apparatus; an oil rig; a petrochemical processing, storage or transport apparatus; a vehicle or a container.

Optionally, the environment is an ex vivo bodily fluid (e.g., urine, blood, blood product, sweat, tears, sputum or spit), bodily solid (e.g., faeces) or tissue of a human or animal subject that has been administered the composition.

Optionally, the environment is an in vivo bodily fluid (e.g., urine, blood, blood product, sweat, tears, sputum or spit), bodily solid (e.g., faeces) or tissue of a human or animal subject that has been administered the composition.

Optionally, the toxic agent comprises one or more components of a CRISPR/Cas system, eg, a DNA sequence encoding one or more components of Type I Cascade (eg, CasA).

Optionally, the toxic agent comprises a DNA sequence encoding guided nuclease, such as a Cas nuclease, TALEN, zinc finger nuclease or meganuclease.

In an example, the carrier cell(s) or composition are comprised by a medical container, eg, a syringe, vial, IV bag, inhaler, eye dropper or nebulizer. In an example, the carrier cell(s) or composition are comprised by a sterile container. In an example, the carrier cell(s) or composition are comprised by a medically-compatible container. In an example, the carrier cell(s) or composition are comprised by a fermentation vessel, eg, a metal, glass or plastic vessel. In an example, the carrier cell(s) or composition are comprised by an agricultural apparatus. In an example, the carrier cell(s) or composition are comprised by food production or processing apparatus. In an example, the carrier cell(s) or composition are comprised by a horticultural apparatus. In an example, the carrier cell(s) or composition are comprised by a farming apparatus. In an example, the carrier cell(s) or composition are comprised by petrochemicals recovery or processing apparatus. In an example, the carrier cell(s) or composition are comprised by a distillation apparatus. In an example, the carrier cell(s) or composition are comprised by cell culture vessel (eg, having a capacity of at least 50, 100, 1000, 10000 or 100000 litres). Additionally or alternatively, the target cell(s) are comprised by any of these apparatus etc.

In an example, the carrier cell(s) or composition are comprised by a medicament, e,g in combination with instructions or a packaging label with directions to administer the medicament by oral, IV, subcutaneous, intranasal, intraocular, vaginal, topical, rectal or inhaled administration to a human or animal subject. In an example, the carrier cell(s) or composition are comprised by an oral medicament formulation. In an example, the carrier cell(s) or composition are comprised by an intranasal or ocular medicament formulation. In an example, the carrier cell(s) or composition are comprised by a personal hygiene composition (eg, shampoo, soap or deodorant) or cosmetic formulation. In an example, th the carrier cell(s) or composition are comprised by a detergent formulation. In an example, the carrier cell(s) or composition are comprised by a cleaning formulation, eg, for cleaning a medical or industrial device or apparatatus. In an example, the carrier cell(s) or composition are comprised by foodstuff, foodstuff ingredient or foodstuff processing agent. In an example, the carrier cell(s) or composition are comprised by beverage, beverage ingredient or beverage processing agent. In an example, the carrier cell(s) or composition are comprised by a medical bandage, fabric, plaster or swab. In an example, the carrier cell(s) or composition are comprised by a herbicide or pesticide. In an example, the carrier cell(s) or composition are comprised by an insecticide.

In an example, the CRISPR/Cas component(s) are component(s) of a Type I CRISPR/Cas system. In an example, the CRISPR/Cas component(s) are component(s) of a Type II CRISPR/Cas system. In an example, the CRISPR/Cas component(s) are component(s) of a Type III CRISPR/Cas system. In an example, the CRISPR/Cas component(s) are component(s) of a Type IV CRISPR/Cas system. In an example, the CRISPR/Cas component(s) are component(s) of a Type V CRISPR/Cas system. In an example, the CRISPR/Cas component(s) comprise a Cas9-encoding nucleotide sequence (eg, S. pyogenes Cas9, S. aureus Cas9 or S. thermophilus Cas9). In an example, the CRISPR/Cas component(s) comprise a Cas3-encoding nucleotide sequence (eg, E. coli Cas3, C. dificile Cas3 or Salmonella Cas3). In an example, the CRISPR/Cas component(s) comprise a Cpf-encoding nucleotide sequence. In an example, the CRISPR/Cas component(s) comprise a CasX-encoding nucleotide sequence. In an example, the CRISPR/Cas component(s) comprise a CasY-encoding nucleotide sequence.

In an example, each carrier cell encodes a CRISPR/Cas component or protein of interest from a nucleotide sequence (NSI) comprising a promoter that is operable in the target bacteria.

Optionally, target bacteria are gram negative bacteria (eg, a spirilla or vibrio). Optionally, target bacteria are gram positive bacteria. Optionally, target bacteria are mycoplasma, chlamydiae, spirochete or mycobacterium bacteria. Optionally, target bacteria are Streptococcus (eg, pyogenes or thermophilus). Optionally, target bacteria are Staphylococcus (eg, aureus, eg, MRSA). Optionally, target bacteria are E. coli (eg, O157: H7), eg, wherein the Cas is encoded by the vecor or an endogenous target cell Cas nuclease (eg, Cas3) activity is de-repressed. Optionally, target bacteria are Pseudomonas (eg, syringae or aeruginosa). Optionally, target bacteria are Vibro (eg, cholerae (eg, O139) or vulnificus). Optionally, target bacteria are Neisseria (eg, gonnorrhoeae or meningitidis). Optionally, target bacteria are Bordetella (eg, pertussis). Optionally, target bacteria are Haemophilus (eg, influenzae). Optionally, target bacteria are Shigella (eg, dysenteriae). Optionally, target bacteria are Brucella (eg, abortus). Optionally, target bacteria are Francisella host. Optionally, target bacteria are Xanthomonas. Optionally, target bacteria are Agrobacterium. Optionally, target bacteria are Erwinia. Optionally, target bacteria are Legionella (eg, pneumophila). Optionally, target bacteria are Listeria (eg, monocytogenes). Optionally, target bacteria are Campylobacter (eg, jejuni). Optionally, target bacteria are Yersinia (eg, pestis). Optionally, target bacteria are Borelia (eg, burgdorferi). Optionally, target bacteria are Helicobacter (eg, pylori). Optionally, target bacteria are Clostridium (eg, dificile or botulinum). Optionally, target bacteria are Erlichia (eg, chaffeensis). Optionally, target bacteria are Salmonella (eg, typhi or enterica, eg, serotype typhimurium, eg, DT 104). Optionally, target bacteria are Chlamydia (eg, pneumoniae). Optionally, target bacteria are Parachlamydia host. Optionally, target bacteria are Corynebacterium (eg, amycolatum). Optionally, target bacteria are Klebsiella (eg, pneumoniae). Optionally, target bacteria are Enterococcus (eg, faecalis or faecim, eg, linezolid-resistant). Optionally, target bacteria are Acinetobacter (eg, baumannii, eg, multiple drug resistant).

Further examples of target cells are as one of the options that follow:

Optionally the target bacteria are Staphylococcus aureus cells, eg, resistant to an antibiotic selected from methicillin, vancomycin, linezolid, daptomycin, quinupristin, dalfopristin and teicoplanin.

Optionally the target bacteria are Pseudomonas aeuroginosa cells, eg, resistant to an antibiotic selected from cephalosporins (eg, ceftazidime), carbapenems (eg, imipenem or meropenem), fluoroquinolones, aminoglycosides (eg, gentamicin or tobramycin) and colistin.

Optionally the target bacteria are Klebsiella (eg, pneumoniae) cells, eg, resistant to carbapenem.

Optionally the target bacteria are Streptoccocus (eg, thermophilus, pneumoniae or pyogenes) cells, eg, resistant to an antibiotic selected from erythromycin, clindamycin, beta-lactam, macrolide, amoxicillin, azithromycin and penicillin.

Optionally the target bacteria are Salmonella (eg, serotype typhi) cells, eg, resistant to an antibiotic selected from ceftriaxone, azithromycin and ciprofloxacin.

Optionally the target bacteria are Shigella cells, eg, resistant to an antibiotic selected from ciprofloxacin and azithromycin.

Optionally the target bacteria are Mycobacterium tuberculosis cells, eg, resistant to an antibiotic selected from Resistance to isoniazid (INH), rifampicin (RMP), fluoroquinolone, amikacin, kanamycin and capreomycin and azithromycin.

Optionally the target bacteria are Enterococcus cells, eg, resistant to vancomycin.

Optionally the target bacteria are Enterobacteriaceae cells, eg, resistant to an antibiotic selected from a cephalosporin and carbapenem.

Optionally the target bacteria are E. coli cells, eg, resistant to an antibiotic selected from trimethoprim, itrofurantoin, cefalexin and amoxicillin.

Optionally the target bacteria are Clostridium (eg, dificile) cells, eg, resistant to an antibiotic selected from fluoroquinolone antibiotic and carbapenem.

Optionally the target bacteria are Neisseria gonnorrhoea cells, eg, resistant to an antibiotic selected from cefixime (eg, an oral cephalosporin), ceftriaxone (an injectable cephalosporin), azithromycin and tetracycline.

Optionally the target bacteria are Acinetoebacter baumannii cells, eg, resistant to an antibiotic selected from beta-lactam, meropenem and a carbapenem.

Optionally the target bacteria are Campylobacter (eg, jejuni) cells, eg, resistant to an antibiotic selected from ciprofloxacin and azithromycin.

Optionally, the target cell(s) produce Beta (β)-lactamase (eg, ESBL-producing E. coli or ESBL-producing Klebsiella).

For example, the target cell(s) are bacterial cells that are resistant to an antibiotic recited in any one of these options above.

In an example, the target cell(s) is a cell of a species selected from Shigella, E. coli, Salmonella, Serratia, Klebsiella, Yersinia, Pseudomonas and Enterobacter.

Optionally, the composition comprises carrier cells that are each or in combination capable of conjugative transfer of plasmid DNAs into target cells of species selected from two or more of Shigella, E. coli, Salmonella, Serratia, Klebsiella, Yersinia, Pseudomonas and Enterobacter.

In an example, the reduction in growth or proliferation of carrier cells is at least 50, 60, 70, 80, 90 or 95%. Optionally, the composition or carrier cell(s) are administered simultaneously or sequentially with an an antibiotic that is toxic to the target cells. For example, the antibiotic can be any antibiotic disclosed herein.

Optioanlly, the expression of the NSI is under the control of an inducible promoter that is operable in the target cell. Optioanlly, the expression of the NSI is under the control of a constitutive promoterthat is operable in the target cell.

In embodiments, the plasmid DNA contains a screenable or selectable marker gene. For example, the selectable marker gene is an antibiotic resistance gene.

The carrier bacteria can be bacteria of a species or genus selected from those appearing in Table 2. For example, the species is found in warm-blooded animals (eg, livestock vertebrates). For example, the species is found in humans. For example, the species is found in plants. Preferably, non-pathogenic bacteria that colonize the non-sterile parts of the human or animal body (e.g., skin, digestive tract, urogenital region, mouth, nasal passages, throat and upper airway, ears and eyes) are utilized as carrier cells, and in an example the methodology of the invention is used to combat a target cell bacterial infection of such a part of the body of a human or animal. In another embodiment, the infection is systemic infection. Examples of particularly preferred carrier bacterial species include, but are not limited to: non-pathogenic strains of Escherichia coli (E. coli F18 and E. coli strain Nissle), various species of Lactobacillus (such as L. casei, L. plantarum, L. paracasei, L. acidophilus, L. fermentum, L. zeae and L. gasseri), or other nonpathogenic or probiotic skin- or GI colonizing bacteria such as Lactococcus, Bifidobacteria, Eubacteria, and bacterial mini-cells, which are anucleoid cells destined to die but still capable of transferring plasmids (see; e.g., Adler et al., Proc. Natl. Acad. Sci. USA 57; 321-326, 1970; Frazer and Curtiss III, Current Topics in Microbiology and Immunology 69: 1-84, 1975; U.S. Pat. No. 4,968,619 to Curtiss III). In some embodiments, the target recipient cells are pathogenic bacteria comprised by a human, animal or plant, eg, on the skin or in the digestive tract, urogenital region, mouth, nasal passage, throat and upper airway, eye(s) and ear(s). Of particular interest for targeting and eradication are pathogenic strains of Pseudomonas aeruginosa, Escherichia coli, Staphylococcus pneumoniae and other species, Enterobacter spp., Enterococcus spp. and Mycobacterium tuberculosis. In an example, the target cell genus or species is any genus or species listed in Table 2.

The present invention finds use with a wide array of settings or environments, eg, in therapeutic, agricultural, or other settings, including, but not limited to, those described in U.S. Pat. Nos. 6,271,359, 6,261,842, 6,221,582, 6,153,381, 6,106,854, and 5,627,275. Others are also discussed herein, and still others will be readily apparent to those of skill in the art.

Numerous types of plasmids comprising the plasmid DNA are suitable for use in the present invention. In view of this, one of skill in the art will appreciate that a single carrier bacterial strain might harbor more than one type of such plasmid (eg, differing in the antibacterial agent that they encode). Further, in another example two or more different carrier bacterial strains, each containing one or more such plasmids, may be combined for a multi-target effect, ie, for killing two or more different target species or strains, or for killing the cells of the same species or strain of target cell.

The present invention finds utility for treatment of humans and in a variety of veterinary, agronomic, horticultural and food processing applications. For human and veterinary use, and depending on the cell population or tissue targeted for protection, the following modes of administration of the carrier bacteria of the invention are contemplated: topical, oral, nasal, ocular, aural, pulmonary (e.g., via an inhaler), ophthalmic, rectal, urogenital, subcutaneous, intraperitoneal and intravenous. The bacteria may be supplied as a pharmaceutical composition, in a delivery vehicle suitable for the mode of administration selected for the patient being treated. The term “patient” or “subject” as used here refers to humans or animals (animals being particularly useful as models for clinical efficacy of a particular donor strain, for example, or being farmed or livestock animals). Commercially-relevant animals are chicken, turkey, duck, catfish, salmon, cod, herring, lobster, shrimp, prawns, cows, sheep, goats, pigs, goats, geese or rabbits.

For example, to deliver the carrier bacteria to the gastrointestinal tract or to the nasal passages, the preferred mode of administration may be by oral ingestion or nasal aerosol, or by feeding (alone or incorporated into the subject's feed or food and/or beverage, such as drinking water). In this regard, the carrier cells may be comprised by a food of livestock (or farmed or companion animal), eg, the carrier bacteria are comprised by a feed additive for livestock. Alternatively, the additive is a beverage (eg, water) additive for livestock. It should be noted that probiotic bacteria, such as Lactobacillus acidophilus, are sold as gel capsules containing a lyophilized mixture of bacterial cells and a solid support such as mannitol. When the gel capsule is ingested with liquid, the lyophilized cells are re-hydrated and become viable, colonogenic bacteria. Thus, in a similar fashion, carrier bacterial cells of the present invention can be supplied as a powdered, lyophilized preparation in a gel capsule, or in bulk, eg, for sprinkling onto food or beverages. The re-hydrated, viable bacterial cells will then populate and/or colomze sites throughout the upper and/or lower gastrointestinal system, and thereafter come into contact with the target bacteria.

For topical applications, the carrier bacteria may be formulated as an ointment or cream to be spread on the affected skin surface. Ointment or cream formulations are also suitable for rectal or vaginal delivery, along with other standard formulations, such as suppositories. The appropriate formulations for topical, vaginal or rectal administration are well known to medicinal chemists.

The present invention will be of particular utility for topical or mucosal administrations to treat a variety of bacterial infections or bacterially related undesirable conditions. Some representative examples of these uses include treatment of (1) conjunctivitis, caused by Haemophilus sp., and corneal ulcers, caused by Pseudomonas aeruginosa; (2) otititis externa, caused by Pseudomonas aeruginosa; (3) chronic sinusitis, caused by many Gram-positive cocci and Gram-negative rods, or for general decontamination of bronchii; (4) cystic fibrosis, associated with Pseudomonas aeruginosa; (5) enteritis, caused by Helicobacter pylori (eg, to treat or prevent gastric ulcers), Escherichia coli, Salmonella typhimurium, Campylobacter or Shigella sp.; (6) open wounds, such as surgical or non-surgical, eg, as a prophylactic measure; (7) burns to eliminate Pseudomonas aeruginosa or other Gram-negative pathogens; (8) acne, eg, caused by Propionobacter acnes; (9) nose or skin infection, eg, caused by metlncillin resistant Staphylococcus aureus (MSRA); (10) body odor, eg, caused by Gram-positive anaerobic bacteria (i.e., use of carrier cells in deodorants); (11) bacterial vaginosis, eg, associated with Gardnerella vaginalis or other anaerobes; and (12) gingivitis and/or tooth decay caused by various organisms.

In one example, the target cells are E. coli cells and the disease or condition to be treated in a human is a uterine tract infection or a ventilator associated infection, eg, pneumonia.

In other embodiments, the carrier cells of the present invention find application in the treatment of surfaces for the removal or attenuation of unwanted target bacteria, for example use in a method of treating such a surface or an environment comprising target bacteria, wherein the method comprises contacting the surface or environment with carrier bacteria of the invention, allowing conjugative transfer of the plasmid DNA of the invention from the carrier to the target bacteria, and allowing the antibacterial agent to kill target cells. For example, surfaces that may be used in invasive treatments such as surgery, catheterization and the like may be treated to prevent infection of a subject by bacterial contaminants on the surface. It is contemplated that the methods and compositions of the present invention may be used to treat numerous surfaces, objects, materials and the like (e.g., medical or first aid equipment, nursery and kitchen equipment and surfaces) to control bacterial contamination thereon.

Pharmaceutical preparations or other compositions comprising the carrier bacteria may be formulated in dosage unit form for ease of administration and uniformity of dosage. Dosage unit form, as used herein, refers to a physically discrete unit of the pharmaceutical preparation appropriate for the patient or plant or environment or surface undergoing treatment. Each dosage should contain a quantity of the carrier bacteria calculated to produce the desired antibacterial effect in association with the selected carrier. Procedures for determining the appropriate dosage unit are well known to those skilled in the art. Dosage units may be proportionately increased or decreased based on the weight of a patient, plant, surface or environment. Appropriate concentrations for achieving eradication of pathogenic target cells (eg, comprised by a tissue of the patient) may be determined by dosage concentration curve calculations, as known in the art.

Other uses for the carrier bacteria of the invention are also contemplated. These include a variety agricultural, horticultural, environmental and food processing applications. For example, in agriculture and horticulture, various plant pathogenic bacteria may be targeted in order to minimize plant disease. One example of a plant pathogen suitable for targeting is Erwinia (eg, E. amvlovora, the causal agent of fire blight). Similar strategies may be utilized to reduce or prevent wilting of cut flowers. For veterinary or animal farming, the carrier cells of the invention may be incorporated into animal feed (chicken, swine, poultry, goat, sheep, fish, shellfish or cattle feed) to reduce bio-burden or to eliminate certain pathogenic organisms (e.g., Salmonella, such as in chicken, turkey or other poultry). In other embodiments, the invention may be applied on meat or other foods to eliminate unwanted or pathogenic bacteria (e.g., E. coli O157:H7 on meat, or Proteus spp., one cause of “fishy odour” on seafood).

Environmental utilities comprise, for example, engineering carrier bacteria to deliver and conditionally express an insecticidal agent in addition to or instead of an antibacterial agent (e.g., for the control of mosquitos that disseminate malaria or West Nile virus). In such applications, as well as in the agricultural and horticultural or other applications described above, formulation of the carrier bacteria as solutions, aerosols, or gel capsules are contemplated.

As used herein, the term “carrier cell”, “first cell” or “donor cell” includes dividing and/or non-dividing bacterial cells (minicells and maxicells), or conditionally non-functional cells.

In an example, the plasmid is an engineered RK2 or RP4 plasmid. In an example the plasmid DNA is comprised by an engineered RK2/RP4 plasmid (ie, a RK2 plasmid that has been modified by recombinant DNA technology or a progeny of such a modified plasmid). Plasmid RK2 is a promiscuous plasmid that can replicate in 29 (and probably many more) gram-negative species (Guiney and Lanka, 1989, p 27-54. In C. M. Thomas (ed) Promiscous plasmids in gram-negative bacteria. London, Ltd London United Kingdom.). Plasmid RK2 is a 60-kb self-transmissible plasmid with a complete nucleotide sequence known (Pansegrau et al., 1994, J. Mol. Biol. 239, 623-663). A minimal replicon derived from this large plasmid has been obtained that is devoid of all its genes except for a trfA gene, that encodes plasmid's Rep protein called TrfA, and an origin of vegetative replication oriV For a review of RK2 replication and its control by TrfA protein, see Helinski et al., 1996 (In Escherichia coli and Salmonella Cellular and Molecular Biology, Vol. 2 (ed. F. Neidhardt, et al., 2295-2324, ASM Press, Washington D.C.).

In an example the plasmid DNA is comprised by an engineered R6K plasmid (ie, a R6K plasmid that has been modified by recombinant DNA technology or a progeny of such a modified plasmid).

The present invention is optionally for an industrial or domestic use, or is used in a method for such use. For example, it is for or used in agriculture, oil or petroleum industry, food or drink industry, clothing industry, packaging industry, electronics industry, computer industry, environmental industry, chemical industry, aeorspace industry, automotive industry, biotechnology industry, medical industry, healthcare industry, dentistry industry, energy industry, consumer products industry, pharmaceutical industry, mining industry, cleaning industry, forestry industry, fishing industry, leisure industry, recycling industry, cosmetics industry, plastics industry, pulp or paper industry, textile industry, clothing industry, leather or suede or animal hide industry, tobacco industry or steel industry.

The present invention is optionally for use in an industry or the environment is an industrial environment, wherein the industry is an industry of a field selected from the group consisting of the medical and healthcare; pharmaceutical; human food; animal food; plant fertilizers; beverage; dairy; meat processing; agriculture; livestock farming; poultry farming; fish and shellfish farming; veterinary; oil; gas; petrochemical; water treatment; sewage treatment; packaging; electronics and computer; personal healthcare and toiletries; cosmetics; dental; non-medical dental; ophthalmic; non-medical ophthalmic; mineral mining and processing; metals mining and processing; quarrying; aviation; automotive; rail; shipping; space; environmental; soil treatment; pulp and paper; clothing manufacture; dyes; printing; adhesives; air treatment; solvents; biodefence; vitamin supplements; cold storage; fibre retting and production; biotechnology; chemical; industrial cleaning products; domestic cleaning products; soaps and detergents; consumer products; forestry; fishing; leisure; recycling; plastics; hide, leather and suede; waste management; funeral and undertaking; fuel; building; energy; steel; and tobacco industry fields.

In an example, the plasmid DNA comprises a CRISPR array that targets target bacteria, wherein the array comprises one, or two or more different spacers (eg, 2, 3, 4, 5, 6, 7, 8, 9 ,10, 20, 30, 40, 50 or more spacers) for targeting the genome of target bacteria.

In an example, the target bacteria are comprised by an environment as follows. In an example, the environment is a microbiome of a human, eg, the oral cavity microbiome or gut microbiome or the bloodstream. In an example, the environment is not an environment in or on a human In an example, the environment is not an environment in or on a non-human animal. In an embodiment, the environment is an air environment. In an embodiment, the environment is an agricultural environment. In an embodiment, the environment is an oil or petroleum recovery environment, eg, an oil or petroleum field or well. In an example, the environment is an environment in or on a foodstuff or beverage for human or non-human animal consumption. In an example, the environment is a maritimeenvironment, eg, in seawater or on a boat (eg, in ship or boat ballast water).

In an example, the environment is a a human or animal microbiome (eg, gut, vaginal, scalp, armpit, skin or oral cavity microbiome). In an example, the target bacteria are comprised by a human or animal microbiome (eg, gut, vaginal, scalp, armpit, skin or oral cavity microbiome).

In an example, the carrier bacteria or composition of the invention are administered intranasally, topically or orally to a human or non-human animal, or is for such administration. The skilled person aiming to treat a microbiome of the human or animal will be able to determine the best route of administration, depending upon the microbiome of interest. For example, when the microbiome is a gut microbiome, administration can be intranasally or orally. When the microbiome is a scalp or armpit microbiome, administration can be topically. When the microbiome is in the mouth or throat, the administration can be orally.

In an example, the environment is harboured by a beverage or water (eg, a waterway or drinking water for human consumption) or soil. The water is optionally in a heating, cooling or industrial system, or in a drinking water storage container.

In an example, the carrier and/or target bacteraia are Firmicutes selected from Anaerotruncus, Acetanaerobacterium, Acetitomaculum, Acetivibrio, Anaerococcus, Anaerofilum, Anaerosinus, Anaerostipes, Anaerovorax, Butyrivibrio, Clostridium, Capracoccus, Dehalobacter, Dialister, Dorea, Enterococcus, Ethanoligenens, Faecalibacterium, Fusobacterium, Gracilibacter, Guggenheimella, Hespellia, Lachnobacterium, Lachnospira, Lactobacillus, Leuconostoc, Megamonas, Mitsuokella, Oribacterium, Oxobacter, Papillibacter, Proprionispira, Pseudobutyrivibrio, Pseudoramibacter, Roseburia, Ruminococcus, Sarcina, Seinonella, Shuttleworthia, Sporobacter, Sporobacterium, Streptococcus, Subdoligranulum, Syntrophococcus, Thermobacillus, Turibacter and Weisella.

In an example, the carrier bacteria, composition, use or method is for reducing pathogenic infections or for re-balancing gut or oral biofilm eg, for treating or preventing obesity or disease in a human or animal; or for treating or preventing a GI condition (such as Crohn's disease, IBD or colitis). For example, the DNA, carrier bacteria, composition, use or method is for knocking-down Salmomnella, Campylobacter, Erwinia, Xanthomonous, Edwardsiella, Pseudomonas, Klebsiella, Pectobacterium, Clostridium dificile or E. coli bacteria in a gut biofilm of a human or animal or a plant, preferably in a human or animal

In an example, the animal is a chicken, eg, and the target bacteria are Salmomnella or Campylobacter. In an example, the animal is a fish (eg, catfish or salmon) or shellfish (eg, prawn or lobster), eg, and the target bacteria are Edwardsiella. In an example, the plant is a potato plant and, eg, the target bacteria are Pectobacterium. In an example, the plant is a cabbage plant and, eg, the target bacteria are Xanthomonous (eg, X. campestris). In an example, the plant is a marijuana plant and, eg, the targt bacteria are Pseudomonas (eg, P. cannabina or P. amygdali), Agrobacterium (eg, A. tumefaciens) or Xanthomonas (eg, X. campestris). In an example, the plant is a hemp plant and, eg, the targt bacteria are are Pseudomonas (eg, P. cannabina or P. amygdali), Agrobacterium (eg, A. tumefaciens) or Xanthomonas (eg, X. campestris).

In an example, the disease or condition is a cancer, inflammatory or autoimmune disease or condition, eg, obesity, diabetes IBD, a GI tract condition or an oral cavity condition.

Optionally, the environment is comprised by, or the target bacteria are comprised by, a gut biofilm, skin biofilm, oral cavity biofilm, throat biofilm, hair biofilm, armpit biofilm, vaginal biofilm, rectal biofilm, anal biofilm, ocular biofilm, nasal biofilm, tongue biofilm, lung biofilm, liver biofilm, kidney biofilm, genital biofilm, penile biofilm, scrotal biofilm, mammary gland biofilm, ear biofilm, urethra biofilm, labial biofilm, organ biofilm or dental biofilm. Optionally, the environment is comprised by, or the target bacteria are comprised by, a plant (eg, a tobacco, crop plant, fruit plant, vegetable plant or tobacco, eg on the surface of a plant or contained in a plant) or by an environment (eg, soil or water or a waterway or acqueous liquid).

In an example, the carrier cell(s) or composition is for treating a disease or condition in an animal or human. In an example, the disease or condition is caused by or mediated by an infection of target cells comprised by the subject or patient. In an example, the disease or condition is associated with an infection of target cells comprised by the subject or patient. In an example, a symptom of the disease or condition is an infection of target cells comprised by the subject or patient.

Optionally, the disease or condition of a human or animal subject is selected from
(a) A neurodegenerative disease or condition;
(b) A brain disease or condition;
(c) A CNS disease or condition;
(d) Memory loss or impairment;
(e) A heart or cardiovascular disease or condition, eg, heart attack, stroke or atrial fibrillation;
(f) A liver disease or condition;
(g) A kidney disease or condition, eg, chronic kidney disease (CKD);
(h) A pancreas disease or condition;
(i) A lung disease or condition, eg, cystic fibrosis or COPD;
(j) A gastrointestinal disease or condition;
(k) A throat or oral cavity disease or condition;
(l) An ocular disease or condition;
(m) A genital disease or condition, eg, a vaginal, labial, penile or scrotal disease or condition;
(n) A sexually-transmissible disease or condition, eg, gonorrhea, HIV infection, syphilis or Chlamydia infection;
(o) An ear disease or condition;
(p) A skin disease or condition;
(q) A heart disease or condition;
(r) A nasal disease or condition
(s) A haematological disease or condition, eg, anaemia, eg, anaemia of chronic disease or cancer;
(t) A viral infection;
(u) A pathogenic bacterial infection;
(v) A cancer;
(w) An autoimmune disease or condition, eg, SLE;
(x) An inflammatory disease or condition, eg, rheumatoid arthritis, psoriasis, eczema, asthma, ulcerative colitis, colitis, Crohn's disease or IBD;
(y) Autism;
(z) ADHD;
(aa) Bipolar disorder;
(bb) ALS [Amyotrophic Lateral Sclerosis];
(cc) Osteoarthritis;
(dd) A congenital or development defect or condition;
(ee) Miscarriage;
(ff) A blood clotting condition;
(gg) Bronchitis;
(hh) Dry or wet AMD;
(ii) Neovascularisation (eg, of a tumour or in the eye);
(jj) Common cold;
(kk) Epilepsy;
(ll) Fibrosis, eg, liver or lung fibrosis;
(mm) A fungal disease or condition, eg, thrush;
(nn) A metabolic disease or condition, eg, obesity, anorexia, diabetes, Type I or Type II diabetes.
(oo) Ulcer(s), eg, gastric ulceration or skin ulceration;
(pp) Dry skin;
(qq) Sjogren's syndrome;
(rr) Cytokine storm;
(ss) Deafness, hearing loss or impairment;
(tt) Slow or fast metabolism (ie, slower or faster than average for the weight, sex and age of the subject);
(uu) Conception disorder, eg, infertility or low fertility;
(vv) Jaundice;
(ww) Skin rash;
(xx) Kawasaki Disease;
(yy) Lyme Disease;
(zz) An allergy, eg, a nut, grass, pollen, dust mite, cat or dog fur or dander allergy;
(aaa) Malaria, typhoid fever, tuberculosis or cholera;
(bbb) Depression;
(ccc) Mental retardation;
(ddd) Microcephaly;
(eee) Malnutrition;
(fff) Conjunctivitis;
(ggg) Pneumonia;
(hhh) Pulmonary embolism;
(iii) Pulmonary hypertension;
(jjj) A bone disorder;
(kkk) Sepsis or septic shock;
(ll) Sinusitus;
(mmm) Stress (eg, occupational stress);
(nnn) Thalassaemia, anaemia, von Willebrand Disease, or haemophilia;
(ooo) Shingles or cold sore;
(ppp) Menstruation;
(qqq) Low sperm count.

Neurodegenerative or CNS Diseases or Conditions for Treatment or Prevention by the Invention

In an example, the neurodegenerative or CNS disease or condition is selected from the group consisting of Alzheimer disease, geriopsychosis, Down syndrome, Parkinson's disease, Creutzfeldt-jakob disease, diabetic neuropathy, Parkinson syndrome, Huntington's disease, Machado-Joseph disease, amyotrophic lateral sclerosis, diabetic neuropathy, and Creutzfeldt Creutzfeldt-Jakob disease. For example, the disease is Alzheimer disease. For example, the disease is Parkinson syndrome.

In an example, wherein the method of the invention is practised on a human or animal subject for treating a CNS or neurodegenerative disease or condition, the method causes downregulation of Treg cells in the subject, thereby promoting entry of systemic monocyte-derived macrophages and/or Treg cells across the choroid plexus into the brain of the subject, whereby the disease or condition (eg, Alzheimer's disease) is treated, prevented or progression thereof is reduced. In an embodiment the method causes an increase of IFN-gamma in the CNS system (eg, in the brain and/or CSF) of the subject. In an example, the method restores nerve fibre and//or reduces the progression of nerve fibre damage. In an example, the method restores nerve myelin and//or reduces the progression of nerve myelin damage. In an example, the method of the invention treats or prevents a disease or condition disclosed in WO2015136541 and/or the method can be used with any method disclosed in WO2015136541 (the disclosure of this document is incorporated by reference herein in its entirety, eg, for providing disclosure of such methods, diseases, conditions and potential therapeutic agents that can be administered to the subject for effecting treatement and/or prevention of CNS and neurodegenerative diseases and conditions, eg, agents such as immune checkpoint inhibitors, eg, anti-PD-1, anti-PD-L1, anti-TIM3 or other antibodies disclosed therein).

Cancers for Treatment or Prevention by the Method

Cancers that may be treated include tumours that are not vascularized, or not substantially vascularized, as well as vascularized tumours. The cancers may comprise non-solid tumours (such as haematological tumours, for example, leukaemias and lymphomas) or may comprise solid tumours. Types of cancers to be treated with the invention include, but are not limited to, carcinoma, blastoma, and sarcoma, and certain leukaemia or lymphoid malignancies, benign and malignant tumours, and malignancies e.g., sarcomas, carcinomas, and melanomas. Adult tumours/cancers and paediatric tumours/cancers are also included.

Haematologic cancers are cancers of the blood or bone marrow. Examples of haematological (or haematogenous) cancers include leukaemias, including acute leukaemias (such as acute lymphocytic leukaemia, acute myelocytic leukaemia, acute myelogenous leukaemia and myeloblasts, promyeiocytic, myelomonocytic, monocytic and erythroleukaemia), chronic leukaemias (such as chronic myelocytic (granulocytic) leukaemia, chronic myelogenous leukaemia, and chronic lymphocytic leukaemia), polycythemia vera, lymphoma, Hodgkin's disease, non-Hodgkin's lymphoma (indolent and high grade forms), multiple myeloma, Waldenstrom's macroglobulinemia, heavy chain disease, myeiodysplastic syndrome, hairy cell leukaemia and myelodysplasia.

Solid tumours are abnormal masses of tissue that usually do not contain cysts or liquid areas. Solid tumours can be benign or malignant. Different types of solid tumours are named for the type of cells that form them (such as sarcomas, carcinomas, and lymphomas). Examples of solid tumours, such as sarcomas and carcinomas, include fibrosarcoma, myxosarcoma, liposarcoma, chondrosarcoma, osteosarcoma, and other sarcomas, synovioma, mesothelioma, Ewing's tumour, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous eel! carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumour, cervical cancer, testicular tumour, seminoma, bladder carcinoma, melanoma, and CNS tumours (such as a glioma (such as brainstem glioma and mixed gliomas), glioblastoma (also known as glioblastoma multiforme) astrocytoma, CNS lymphoma, germinoma, medu!loblastoma, Schwannoma craniopharyogioma, ependymoma, pineaioma, hemangioblastoma, acoustic neuroma, oligodendroglioma, menangioma, neuroblastoma, retinoblastoma and brain metastases).

Autoimmune Diseases for Treatment or Prevention by the Method
1. Acute Disseminated Encephalomyelitis (ADEM)
2. Acute necrotizing hemorrhagic leukoencephalitis
3. Addison's disease
4. Agammaglobulinemia
5. Alopecia areata
6. Amyloidosis
7. Ankylosing spondylitis
8. Anti-GBM/Anti-TBMI nephritis
9. Antiphospholipid syndrome (APS)
10. Autoimmune angioedema
11. Autoimmune aplastic anemia
12. Autoimmune dysautonomia
13. Autoimmune hepatitis
14. Autoimmune hyperlipidemia
15. Autoimmune immunodeficiency
16. Autoimmune inner ear disease (AIED)
17. Autoimmune myocarditis
18. Autoimmune oophoritis
19. Autoimmune pancreatitis
20. Autoimmune retinopathy
21. Autoimmune thrombocytopenic purpura (ATP)
22. Autoimmune thyroid disease
23. Autoimmune urticaria
24. Axonal & neuronal neuropathies
25. Balo disease
26. Behcet's disease
27. Bullous pemphigoid
28. Cardiomyopathy
29. Castleman disease
30. Celiac disease
31. Chagas disease
32. Chronic fatigue syndrome
33. Chronic inflammatory demyelinating polyneuropathy (GDP)
34. Chronic recurrent multifocal ostomyelitis (CRMO)
35. Churg-Strauss syndrome
36. Cicatricial pemphigoid/benign mucosal pemphigoid
37. Crohn's disease
38. Cogans syndrome
39. Cold agglutinin disease
40. Congenital heart block
41. Coxsackie myocarditis
42. CREST disease
43. Essential mixed cryoglobulinemia
44. Demyelinating neuropathies
45. Dermatitis herpetiformis
46. Dermatomyositis
47. Devic's disease (neuromyelitis optica)
48. Discoid lupus
49. Dressler's syndrome
50. Endometriosis
51. Eosinophilic esophagitis
52. Eosinophilic fasciitis
53. Erythema nodosum
54. Experimental allergic encephalomyelitis
55. Evans syndrome
56. Fibromyalgia
57. Fibrosing alveolitis
58. Giant cell arteritis (temporal arteritis)
59. Giant cell myocarditis
60. Glomerulonephritis
61. Goodpasture's syndrome
62. Granulomatosis with Polyangiitis (GPA) (formerly called Wegener's Granulomatosis)
63. Graves' disease
64. Guillain-Barre syndrome
65. Hashimoto's encephalitis
66. Hashimoto's thyroiditis
67. Hemolytic anemia
68. Henoch-Schonlein purpura
69. Herpes gestationis
70. Hypogammaglobulinemia
71. Idiopathic thrombocytopenic purpura (ITP)
72. IgA nephropathy
73. IgG4-related sclerosing disease
74. Immunoregulatory lipoproteins
75. Inclusion body myositis
76. Interstitial cystitis
77. Juvenile arthritis
78. Juvenile diabetes (Type 1 diabetes)
79. Juvenile myositis
80. Kawasaki syndrome
81. Lambert-Eaton syndrome
82. Leukocytoclastic vasculitis
83. Lichen planus
84. Lichen sclerosus
85. Ligneous conjunctivitis
86. Linear IgA disease (LAD)
87. Lupus (SLE)
88. Lyme disease, chronic
89. Meniere's disease
90. Microscopic polyangiitis
91. Mixed connective tissue disease (MCTD)
92. Mooren's ulcer
93. Mucha-Habermann disease
94. Multiple sclerosis
95. Myasthenia gravis
96. Myositis
97. Narcolepsy
98. Neuromyelitis optica (Devic's)
99. Neutropenia
100. Ocular cicatricial pemphigoid
101. Optic neuritis
102. Palindromic rheumatism
103. PANDAS (Pediatric Autoimmune Neuropsychiatric Disorders Associated with Streptococcus)
104. Paraneoplastic cerebellar degeneration
105. Paroxysmal nocturnal hemoglobinuria (PNH)
106. Parry Romberg syndrome
107. Parsonnage-Turner syndrome
108. Pars planitis (peripheral uveitis)
109. Pemphigus
110. Peripheral neuropathy
111. Perivenous encephalomyelitis
112. Pernicious anemia
113. POEMS syndrome
114. Polyarteritis nodosa
115. Type I, II, & III autoimmune polyglandular syndromes
116. Polymyalgia rheumatica
117. Polymyositis
118. Postmyocardial infarction syndrome
119. Postpericardiotomy syndrome
120. Progesterone dermatitis
121. Primary biliary cirrhosis
122. Primary sclerosing cholangitis
123. Psoriasis
124. Psoriatic arthritis
125. Idiopathic pulmonary fibrosis
126. Pyoderma gangrenosum
127. Pure red cell aplasia
128. Raynauds phenomenon
129. Reactive Arthritis
130. Reflex sympathetic dystrophy
131. Reiter's syndrome
132. Relapsing polychondritis
133. Restless legs syndrome
134. Retroperitoneal fibrosis
135. Rheumatic fever
136. Rheumatoid arthritis
137. Sarcoidosis
138. Schmidt syndrome
139. Scleritis
140. Scleroderma
141. Sjogren's syndrome
142. Sperm & testicular autoimmunity
143. Stiff person syndrome
144. Subacute bacterial endocarditis (SBE)
145. Susac's syndrome
146. Sympathetic ophthalmia
147. Takayasu's arteritis
148. Temporal arteritis/Giant cell arteritis
149. Thrombocytopenic purpura (TIP)
150. Tolosa-Hunt syndrome
151. Transverse myelitis
152. Type 1 diabetes
153. Ulcerative colitis
154. Undifferentiated connective tissue disease (UCTD)
155. Uveitis
156. Vasculitis
157. Vesiculobullous dermatosis
158. Vitiligo
159. Wegener's granulomatosis termed Granulomatosis with Polyangiitis (GPA).

Inflammatory Diseases for Treatment or Prevention by the Method
1. Alzheimer's disease
2. ankylosing spondylitis
3. arthritis (osteoarthritis, rheumatoid arthritis (RA), psoriatic arthritis)
4. asthma
5. atherosclerosis
6. Crohn's disease
7. colitis
8. dermatitis
9. diverticulitis
10. fibromyalgia
11. hepatitis
12. irritable bowel syndrome (IBS)
13. systemic lupus erythematous (SLE)
14. nephritis
15. Parkinson's disease
16. ulcerative colitis.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine study, numerous equivalents to the specific procedures described herein. Such equivalents are considered to be within the scope of this invention and are covered by the claims. All publications and patent applications mentioned in the specification are indicative of the level of skill of those skilled in the art to which this invention pertains. All publications and patent applications and all US equivalent patent applications and patents are herein incorporated by reference to the same extent as if each individual publication or patent application was specifically and individually indicated to be incorporated by reference. The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps

The term “or combinations thereof” or similar as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

Any part of this disclosure may be read in combination with any other part of the disclosure, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### EXAMPLES

### Example 1

### Knock-Out of hypC2 Function Greatly Enhances Coniugtion Efficiency

Transposon library construction and enrichment of highly conjugative mutants A transposon mutant library of plasmid pX1.0 (Genbank accession: HM114226.1) was constructed using the EZ-Tn5™ kit (Lucigen, Middleton, USA) following the manufacturer's protocol. E. coli Top10 competent cells (ThermoFisher, Massachusetts, USA) were transformed via electroporation with 1 μl of the library and recovered for 2 h at 37° C. with shaking. The transformation mixture was then diluted five-fold in 50 ug/ml Kanamycin to enrich for plasmids with successful Tn5 transpositions.

Determining Conjugation Efficiency

Overnight cultures of donor (E. coli MG1655 containing wildtype or mutant plasmid) and streptomycin resistant recipient E. coli MG1655 were mixed 1:1 and spotted on the surface of an LB agar plate. The conjugating mixture was incubated for 14 h after which the colony, consisting of the two strains, was scraped of the plate and diluted in PBS. Different dilutions were plated on selective plates to separate and quantify transconjugants, donors, and recipients.

The sequence of hypC2 and its protein are shown in Table 3.

Results of Conjugation Experiment:

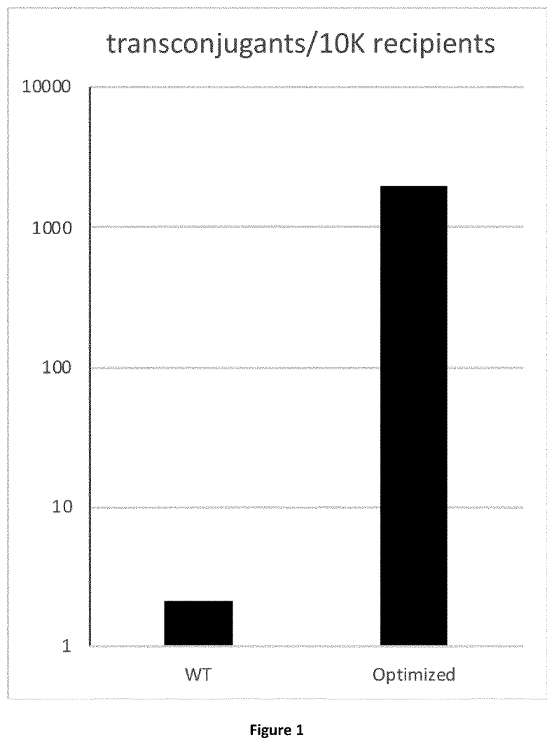

See FIG. 1. The hypc2 KO (knock-out) plasmid mutant shows an approximately 1000× higher conjugation rate compared to the wildtype (WT). Showed is the number of transconjugants (infected cells) obtained from the same amount of donor and incubation time with the WT and optimized (mutant) plasmids.

### Example 2

### hypC2 is Widespread Amongst Plasmids

The pX1.0 plasmid is an archetypical IncX plasmid constructed from naturally occurring IncX plasmids to contain the core functionalities within the IncX plasmid group. See PLoS One. 2011; 6(5):e19912. doi: 10.1371/journal.pone.0019912. Epub 2011 May 18; “Design and synthesis of a quintessential self-transmissible IncX1 plasmid, pX1.0”, Hansen LH et al. IncX plasmids are historically defined by their replication module, which is used in their typing as described elsewhere. See, for example, Plasmid. 2012 July; 68(1):43-50. doi: 10.1016/j.plasmid.2012.03.001. Epub 2012 Mar. 26; “Expansion of the IncX plasmid family for improved identification and typing of novel plasmids in drug-resistant Enterobacteriaceae”, Johnson T J et al.

A blast of the hypC2 gene reveals that identical (100% ID and coverage) genes are present in 33 different plasmids in Genbank. Apart from these it is found in 68 sequenced plasmids with more than 80% coverage and 80% ID, after which a steep drop in both ID and coverage (to unrelated sequences) is observed. The gene is found in E. coli and Salmonella species, but also in Shigella and Klebsiella at high identity.

As in pairwise alignment output:

% ID it the percentage of (perfect) matches in the aligment and (query) coverage is the percentage (of the length) of the query (hyp2C) sequence aligning to the target sequence.

TABLE 1
Relatedness of hvpC2 found in Individual examples of
plasmids found in different bacterial host species:

| Accession no | ID % | Coverage % | Plasmid | Species | SEQ ID NO: |
|---|---|---|---|---|---|
| HG963477.1 | 100 | 100 | pIS15_43 | E. coli | 1 |
| CP023137.2 | 96.7 | 98 | Unnamed | K. pneumoniae | 3 |
| CP005391.2 | 96.6 | 100 | pCFSAN002069 | S. enterica | 4 |
| CP013972.1 | 79.6 | 99 | Unnamed | E. tracheiphila | 5 |
| CP017588.1 | 98.1 | 99 | pDSJ07 | P. stewartii | 6 |
| CP026699.1 | 78.5 | 88 | Unamed | C. koseri | 7 |

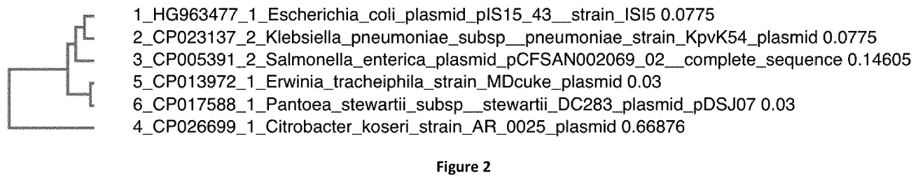

And a phylogram to illustrate is shown in FIG. 2.

TABLE 2
Example Bacteria
Optionally, the carrier (donor or first) cells are selected from this Table and/or the target (recipient or second) cells are selected from this Table (eg, wherein the carrier and target cells are of a different species; or of the same species but are a different strain or the carrier cells are engineered but the target cells are wild-type or vice versa). For example the carrier cells are E coli cells and the target cells are C dificile, E coli, Akkermansia, Enterobacteriacea, Ruminococcus, Faecalibacterium, Firmicutes, Bacteroidetes, Salmonella, Klebsiella, Pseudomonas, Acintenobacter or Streptococcus cells.

| | | | | |
|---|---|---|---|---|
| Abiotrophia | Acidocella | Actinomyces | Alkalilimnicola | Aquaspirillum |
| Abiotrophia | Acidocella | Actinomyces bovis | Alkalilimnicola | Aquaspirillum |
| defectiva | aminolytica | Actinomyces | ehrlichii | polymorphum |
| Acaricomes | Acidocella facilis | denticolens | Alkaliphilus | Aquaspirillum |
| Acaricomes | Acidomonas | Actinomyces | Alkaliphilus | putridiconchylium |
| phytoseiuli | Acidomonas | europaeus | oremlandii | Aquaspirillum |
| Acetitomaculum | methanolica | Actinomyces | Alkaliphilus | serpens |
| Acetitomaculum | Acidothermus | georgiae | transvaalensis | Aquimarina |
| ruminis | Acidothermus | Actinomyces | Allochromatium | Aquimarina |
| Acetivibrio | cellulolyticus | gerencseriae | Allochromatium | latercula |
| Acetivibrio | Acidovorax | Actinomyces | vinosum | Arcanobacterium |
| cellulolyticus | Acidovorax | hordeovulneris | Alloiococcus | Arcanobacterium |
| Acetivibrio | anthurii | Actinomyces | Alloiococcus otitis | haemolyticum |
| ethanolgignens | Acidovorax caeni | howellii | Allokutzneria | Arcanobacterium |
| Acetivibrio | Acidovorax | Actinomyces | Allokutzneria albata | pyogenes |
| multivorans | cattleyae | hyovaginalis | Altererythrobacter | Archangium |
| Acetoanaerobium | Acidovorax citrulli | Actinomyces | Altererythrobacter | Archangium |
| Acetoanaerobium | Acidovorax | israelii | ishigakiensis | gephyra |
| noterae | defluvii | Actinomyces | Altermonas | Arcobacter |
| Acetobacter | Acidovorax | johnsonii | Altermonas | Arcobacter butzleri |
| Acetobacter aceti | delafieldii | Actinomyces | haloplanktis | Arcobacter |
| Acetobacter | Acidovorax facilis | meyeri | Altermonas | cryaerophilus |
| cerevisiae | Acidovorax | Actinomyces | macleodii | Arcobacter |
| Acetobacter | konjaci | naeslundii | Alysiella | halophilus |
| cibinongensis | Acidovorax | Actinomyces neuii | Alysiella crassa | Arcobacter |
| Acetobacter | temperans | Actinomyces | Alysiella filiformis | nitrofigilis |
| estunensis | Acidovorax | odontolyticus | Aminobacter | Arcobacter |
| Acetobacter | valerianellae | Actinomyces oris | Aminobacter | skirrowii |
| fabarum | Acinetobacter | Actinomyces | aganoensis | Arhodomonas |
| Acetobacter | Acinetobacter | radingae | Aminobacter | Arhodomonas |
| ghanensis | baumannii | Actinomyces | aminovorans | aquaeolei |
| Acetobacter | Acinetobacter | slackii | Aminobacter | Arsenophonus |
| indonesiensis | baylyi | Actinomyces | niigataensis | Arsenophonus |
| Acetobacter | Acinetobacter | turicensis | Aminobacterium | nasoniae |
| lovaniensis | bouvetii | Actinomyces | Aminobacterium | Arthrobacter |
| Acetobacter | Acinetobacter | viscosus | mobile | Arthrobacter agilis |
| malorum | calcoaceticus | Actinoplanes | Aminomonas | Arthrobacter albus |
| Acetobacter | Acinetobacter | Actinoplanes | Aminomonas | Arthrobacter |
| nitrogenifigens | gerneri | auranticolor | paucivorans | aurescens |
| Acetobacter oeni | Acinetobacter | Actinoplanes | Ammoniphilus | Arthrobacter |
| Acetobacter | haemolyticus | brasiliensis | Ammoniphilus | chlorophenolicus |
| orientalis | Acinetobacter | Actinoplanes | oxalaticus | Arthrobacter |
| Acetobacter | johnsonii | consettensis | Ammoniphilus | citreus |
| orleanensis | Acinetobacter junii | Actinoplanes | oxalivorans | Arthrobacter |
| Acetobacter | Acinetobacter | deccanensis | Amphibacillus | crystallopoietes |
| pasteurianus | lwoffi | Actinoplanes | Amphibacillus | Arthrobacter |
| Acetobacter | Acinetobacter | derwentensis | xylanus | cumminsii |
| pornorurn | parvus | Actinoplanes | Amphritea | Arthrobacter |
| Acetobacter | Acinetobacter | digitatis | Amphritea balenae | globiformis |
| senegalensis | radioresistens | Actinoplanes | Amphritea japonica | Arthrobacter |
| Acetobacter | Acinetobacter | durhamensis | Amycolatopsis | histidinolovorans |
| xylinus | schindleri | Actinoplanes | Amycolatopsis alba | Arthrobacter ilicis |
| Acetobacterium | Acinetobacter soli | ferrugineus | Amycolatopsis | Arthrobacter luteus |
| Acetobacterium | Acinetobacter | Actinoplanes | albidoflavus | Arthrobacter |
| bakii | tandoii | globisporus | Amycolatopsis | methylotrophus |
| Acetobacterium | Acinetobacter | Actinoplanes | azurea | Arthrobacter |
| carbinolicum | tjernbergiae | humidus | Amycolatopsis | mysorens |
| Acetobacterium | Acinetobacter | Actinoplanes | coloradensis | Arthrobacter |
| dehalogenans | towneri | italicus | Amycolatopsis | nicotianae |
| Acetobacterium | Acinetobacter | Actinoplanes | lurida | Arthrobacter |
| fimetarium | ursingii | liguriensis | Amycolatopsis | nicotinovorans |
| Acetobacterium | Acinetobacter | Actinoplanes | mediterranei | Arthrobacter |
| malicum | venetianus | lobatus | Amycolatopsis | oxydans |
| Acetobacterium | Acrocarpospora | Actinoplanes | rifamycinica | Arthrobacter |
| paludosum | Acrocarpospora | missouriensis | Amycolatopsis | pascens |
| Acetobacterium | corrugata | Actinoplanes | rubida | Arthrobacter |
| tundrae | Acrocarpospora | palleronii | Amycolatopsis | phenanthrenivorans |
| Acetobacterium | macrocephala | Actinoplanes | sulphurea | Arthrobacter |
| wieringae | Acrocarpospora | philippinensis | Amycolatopsis | polychromogenes |
| Acetobacterium | pleiomorpha | Actinoplanes | tolypomycina | Atrhrobacter |
| woodii | Actibacter | rectilineatus | Anabaena | protophormiae |
| Acetofilamentum | Actibacter | Actinoplanes | Anabaena cylindrica | Arthrobacter |
| Acetofilamentum | sediminis | regularis | Anabaena flosaquae | psychrolactophilus |
| rigidum | Actinoalloteichus | Actinoplanes | Anabaena variabilis | Arthrobacter |
| Acetohalobium | Actinoalloteichus | teichomyceticus | Anaeroarcus | ramosus |
| Acetohalobium | cyanogriseus | Actinoplanes | Anaeroarcus | Arthrobacter |
| arabaticum | Actinoalloteichus | utahensis | burkinensis | sulfonivorans |
| Acetomicrobium | hymeniacidonis | Actinopolyspora | Anaerobaculum | Arthrobacter |
| Acetomicrobium | Actinoalloteichus | Actinopolyspora | Anaerobaculum | sulfureus |
| faecale | spitiensis | halophila | mobile | Arthrobacter |
| Acetomicrobium | Actinobaccillus | Actinopolyspora | Anaerobiospirillum | uratoxydans |
| flavidum | Actinobacillus | mortivallis | Anaerobiospirillum | Arthrobacter |
| Acetonema | capsulatus | Actinosynnema | succiniciproducens | ureafaciens |
| Acetonema longum | Actinobacillus | Actinosynnema | Anaerobiospirillum | Arthrobacter |
| Acetothermus | delphinicola | mirum | thomasii | viscosus |
| Acetothermus | Actinobacillus | Actinotalea | Anaerococcus | Arthrobacter |
| paucivorans | hominis | Actinotalea | Anaerococcus | woluwensis |
| Acholeplasma | Actinobacillus | fermentans | hydrogenalis | Asaia |
| Acholeplasma | indolicus | Aerococcus | Anaerococcus | Asaia bogorensis |
| axanthum | Actinobacillus | Aerococcus | lactolyticus | Asanoa |
| Acholeplasma | lignieresii | sanguinicola | Anaerococcus | Asanoa ferruginea |
| brassicae | Actinobacillus | Aerococcus urinae | prevotii | Asticcacaulis |
| Acholeplasma | minor | Aerococcus | Anaerococcus | Asticcacaulis |
| cavigenitalium | Actinobacillus | urinaeequi | tetradius | biprosthecium |
| Acholeplasma | muris | Aerococcus | Anaerococcus | Asticcacaulis |
| equifetale | Actinobacillus | urinaehominis | vaginalis | excentricus |
| Acholeplasma | pleuropneumoniae | Aerococcus | Anaerofustis | Atopobacter |
| granularum | Actinobacillus | viridans | Anaerofustis | Atopobacter |
| Acholeplasma | porcinus | Aeromicrobium | stercorihominis | phocae |
| hippikon | Actinobacillus | Aeromicrobium | Anaeromusa | Atopobium |
| Acholeplasma | rossii | erythreum | Anaeromusa | Atopobium fossor |
| laidlawii | Actinobacillus | Aeromonas | acidaminophila | Atopobium |
| Acholeplasma | scotiae | Aeromonas | Anaeromyxobacter | minutum |
| modicum | Actinobacillus | allosaccharophila | Anaeromyxobacter | Atopobium |
| Acholeplasma | seminis | Aeromonas | dehalogenans | parvulum |
| morum | Actinobacillus | bestiarum | Anaerorhabdus | Atopobium rimae |
| Acholeplasma | succinogenes | Aeromonas caviae | Anaerorhabdus | Atopobium vaginae |
| multilocale | Actinobaccillus | Aeromonas | furcosa | Aureobacterium |
| Acholeplasma | suis | encheleia | Anaerosinus | Aureobacterium |
| oculi | Actinobacillus | Aeromonas | Anaerosinus | barkeri |
| Acholeplasma | ureae | enteropelogenes | glycerini | Aurobacterium |
| palmae | Actinobaculum | Aeromonas | Anaerovirgula | Aurobacterium |
| Acholeplasma | Actinobaculum | eucrenophila | Anaerovirgula | liquefaciens |
| parvum | massiliense | Aeromonas | multivorans | Avibacterium |
| Acholeplasma | Actinobaculum | ichthiosmia | Ancalomicrobium | Avibacterium avium |
| pleciae | schaalii | Aeromonas | Ancalomicrobium | Avibacterium |
| Acholeplasma | Actinobaculum | jandaei | adetum | gallinarum |
| vituli | suis | Aeromonas media | Ancylobacter | Avibacterium |
| Achromobacter | Actinomyces | Aeromonas | Ancylobacter | paragallinarum |
| Achromobacter | urinale | popoffii | aquaticus | Avibacterium |
| denitrificans | Actinocatenispora | Aeromonas sobria | Aneurinibacillus | volantium |
| Achromobacter | Actinocatenispora | Aeromonas veronii | Aneurinibacillus | Azoarcus |
| insolitus | rupis | Agrobacterium | aneurinilyticus | Azoarcus indigens |
| Achromobacter | Actinocatenispora | Agrobacterium | Aneurinibacillus | Azoarcus |
| piechaudii | thailandica | gelatinovorum | migulanus | tolulyticus |
| Achromobacter | Actinocatenispora | Agrococcus | Aneurinibacillus | Azoarcus |
| ruhlandii | sera | Agrococcus | thermoaerophilus | toluvorans |
| Achromobacter | Actinocorallia | citreus | Angiococcus | Azohydromonas |
| spanius | Actinocorallia | Agrococcus | Angiococcus | Azohydromonas |
| Acidaminobacter | aurantiaca | jenensis | disciformis | australica |
| Acidaminobacter | Actinocorallia | Agromonas | Angulomicrobium | Azohydromonas |
| hydrogenoformans | aurea | Agromonas | Angulomicrobium | lata |
| Acidaminococcus | Actinocorallia | oligotrophica | tetraedrale | Azomonas |
| Acidaminococcus | cavernae | Agromyces | Anoxybacillus | Azomonas agilis |
| fermentans | Actinocorallia | Agromyces | Anoxybacillus | Azomonas insignis |
| Acidaminococcus | glomerata | fucosus | pushchinoensis | Azomonas |
| intestini | Actinocorallia | Agromyces | Aquabacterium | macrocytogenes |
| Acidicaldus | herbida | hippuratus | Aquabacterium | Azorhizobium |
| Acidicaldus | Actinocorallia | Agromyces | commune | Azorhizobium |
| organivorans | libanotica | luteolus | Aquabacterium | caulinodans |
| Acidimicrobium | Actinocorallia | Agromyces | parvum | Azorhizophilus |
| Acidimicrobium | longicatena | mediolanus | | Azorhizophilus |
| ferrooxidans | Actinomadura | Agromyces | | paspali |
| Acidiphilium | Actinomadura alba | ramosus | | Azospirillum |
| Acidiphilium | Actinomadura | Agromyces | | Azospirillum |
| acidophilum | atramentaria | rhizospherae | | brasilense |
| Acidiphilium | Actinomadura | Akkermansia | | Azospirillum |
| angustum | bangladeshensis | Akkermansia | | halopraeferens |
| Acidiphilium | Actinomadura | muciniphila | | Azospirillum |
| cryptum | catellatispora | Albidiferax | | irakense |
| Acidiphilium | Actinomadura | Albidiferax | | Azotobacter |
| multivorum | chibensis | ferrireducens | | Azotobacter |
| Acidiphilium | Actinomadura | Albidovulum | | beijerinckii |
| organovorum | chokoriensis | Albidovulum | | Azotobacter |
| Acidiphilium | Actinomadura | inexpectatum | | chroococcum |
| rubrum | citrea | Alcaligenes | | Azotobacter |
| Acidisoma | Actinomadura | Alcaligenes | | nigricans |
| Acidisoma | coerulea | denitrificans | | Azotobacter |
| sibiricum | Actinomadura | Alcaligenes | | salinestris |
| Acidisoma tundrae | echinospora | faecalis | | Azotobacter |
| Acidisphaera | Actinomadura | Alcanivorax | | vinelandii |
| Acidisphaera | fibrosa | Alcanivorax | | |
| rubrifaciens | Actinomadura | borkumensis | | |
| Acidithiobacillus | formosensis | Alcanivorax | | |
| Acidithiobacillus | Actinomadura | jadensis | | |
| albertensis | hibisca | Algicola | | |
| Acidithiobacillus | Actinomadura | Algicola | | |
| caldus | kijaniata | bacteriolytica | | |
| Acidithiobacillus | Actinomadura | Alicyclobacillus | | |
| ferrooxidans | latina | Alicyclobacillus | | |
| Acidithiobacillus | Actinomadura | disulfidooxidans | | |
| thiooxidans | livida | Alicyclobacillus | | |
| Acidobacterium | Actinomadura | sendaiensis | | |
| Acidobacterium | luteofluorescens | Alicyclobacillus | | |
| capsulatum | Actinomadura | vulcanalis | | |
| | macra | Alishewanella | | |
| | Actinomadura | Alishewanella | | |
| | madurae | fetalis | | |
| | Actinomadura | Alkalibacillus | | |
| | oligospora | Alkalibacillus | | |
| | Actinomadura | haloalkaliphilus | | |
| | pelletieri | | | |
| | Actinomadura | | | |
| | rubrobrunea | | | |
| | Actinomadura | | | |
| | rugatobispora | | | |
| | Actinomadura | | | |
| | umbrina | | | |
| | Actinomadura | | | |
| | verrucosospora | | | |
| | Actinomadura | | | |
| | vinacea | | | |
| | Actinomadura | | | |
| | viridilutea | | | |
| | Actinomadura | | | |
| | viridis | | | |
| | Actinomadura | | | |
| | yumaensis | | | |

| | | | | |
|---|---|---|---|---|
| Bacillus | Bacteroides | Bibersteinia | Borrelia | Brevinema |
| [see below] | Bacteroides | Bibersteinia trehalosi | Borrelia afzelii | Brevinema |
| Bacteriovorax | caccae | Bifidobacterium | Borrelia americana | andersonii |
| Bacteriovorax | Bacteroides | Bifidobacterium | Borrelia | Brevundimonas |
| stolpii | coagulans | adolescentis | burgdorferi | Brevundimonas |
| | Bacteroides | Bifidobacterium | Borrelia | alba |
| | eggerthii | angulatum | carolinensis | Brevundimonas |
| | Bacteroides | Bifidobacterium | Borrelia coriaceae | aurantiaca |
| | fragilis | animalis | Borrelia garinii | Brevundimonas |
| | Bacteroides | Bifidobacterium | Borrelia japonica | diminuta |
| | galacturonicus | asteroides | Bosea | Brevundimonas |
| | Bacteroides | Bifidobacterium | Bosea | intermedia |
| | helcogenes | bifidum | minatitlanensis | Brevundimonas |
| | Bacteroides | Bifidobacterium boum | Bosea thiooxidans | subvibrioides |
| | ovatus | Bifidobacterium breve | Brachybacterium | Brevundimonas |
| | Bacteroides | Bifidobacterium | Brachybacterium | vancanneytii |
| | pectinophilus | catenulatum | alimentarium | Brevundimonas |
| | Bacteroides | Bifidobacterium | Brachybacterium | variabilis |
| | pyogenes | choerinum | faecium | Brevundimonas |
| | Bacteroides | Bifidobacterium | Brachybacterium | vesicularis |
| | salyersiae | coryneforme | paraconglomeratum | Brochothrix |
| | Bacteroides | Bifidobacterium | Brachybacterium | Brochothrix |
| | stercoris | cuniculi | rhamnosum | campestris |
| | Bacteroides suis | Bifidobacterium | Brachybacterium | Brochothrix |
| | Bacteroides | dentium | tyrofermentans | thermosphacta |
| | tectus | Bifidobacterium | Brachyspira | Brucella |
| | Bacteroides | gallicum | Brachyspira | Brucella canis |
| | thetaiotaomicron | Bifidobacterium | alvinipulli | Brucella |
| | Bacteroides | gallinarum | Brachyspira | neotomae |
| | uniformis | Bifidobacterium | hyodysenteriae | Bryobacter |
| | Bacteroides | indicum | Brachyspira | Bryobacter |
| | ureolyticus | Bifidobacterium longum | innocens | aggregatus |
| | Bacteroides | Bifidobacterium | Brachyspira | Burkholderia |
| | vulgatus | magnumBifidobacterium | murdochii | Burkholderia |
| | Balnearium | merycicum | Brachyspira | ambifaria |
| | Balnearium | Bifidobacterium | pilosicoli | Burkholderia |
| | lithotrophicum | minimum | Bradyrhizobium | andropogonis |
| | Balneatrix | Bifidobacterium | Bradyrhizobium | Burkholderia |
| | Balneatrix alpica | pseudocatenulatum | canariense | anthina |
| | Balneola | Bifidobacterium | Bradyrhizobium | Burkholderia |
| | Balneola vulgaris | pseudolongum | elkanii | caledonica |
| | Barnesiella | Bifidobacterium | Bradyrhizobium | Burkholderia |
| | Barnesiella | pullorum | japonicum | caryophylli |
| | viscericola | Bifidobacterium | Bradyrhizobium | Burkholderia |
| | Bartonella | ruminantium | liaoningense | cenocepacia |
| | Bartonella | Bifidobacterium | Brenneria | Burkholderia |
| | alsatica | saeculare | Brenneria alni | cepacia |
| | Bartonella | Bifidobacterium subtile | Brenneria | Burkholderia |
| | bacilliformis | Bifidobacterium | nigrifluens | cocovenenans |
| | Bartonella | thermophilum | Brenneria quercina | Burkholderia |
| | clarridgeiae | Bilophila | Brenneria quercina | dolosa |
| | Bartonella | Bilophila wadsworthia | Brenneria salicis | Burkholderia |
| | doshiae | Biostraticola | Brevibacillus | fungorum |
| | Bartonella | Biostraticola tofi | Brevibacillus agri | Burkholderia |
| | elizabethae | Bizionia | Brevibacillus | glathei |
| | Bartonella | Bizionia argentinensis | borstelensis | Burkholderia |
| | grahamii | Blastobacter | Brevibacillus brevis | glumae |
| | Bartonella | Blastobacter capsulatus | Brevibacillus | Burkholderia |
| | henselae | Blastobacter | centrosporus | graminis |
| | Bartonella | denitrificans | Brevibacillus | Burkholderia |
| | rochalimae | Blastococcus | choshinensis | kururiensis |
| | Bartonella | Blastococcus | Brevibacillus | Burkholderia |
| | vinsonii | aggregatus | invocatus | multivorans |
| | Bavariicoccus | Blastococcus | Brevibacillus | Burkholderia |
| | Bavariicoccus | saxobsidens | laterosporus | phenazinium |
| | seileri | Blastochloris | Brevibacillus | Burkholderia |
| | Bdellovibrio | Blastochloris | parabrevis | plantarii |
| | Bdellovibrio | viridis | Brevibacillus | Burkholderia |
| | bacteriovorus | Blastomonas | reuszeri | pyrrocinia |
| | Bdellovibrio | Blastomonas | Brevibacterium | Burkholderia |
| | exovorus | natatoria | Brevibacterium | silvatlantica |
| | Beggiatoa | Blastopirellula | abidum | Burkholderia |
| | Beggiatoa alba | Blastopirellula | Brevibacterium | stabilis |
| | Beijerinckia | marina | album | Burkholderia |
| | Beijerinckia | Blautia | Brevibacterium | thailandensis |
| | derxii | Blautia coccoides | aurantiacum | Burkholderia |
| | Beijerinckia | Blautia hansenii | Brevibacterium | tropica |
| | fluminensis | Blautia producta | celere | Burkholderia |
| | Beijerinckia | Blautia wexlerae | Brevibacterium | unamae |
| | indica | Bogoriella | epidermidis | Burkholderia |
| | Beijerinckia | Bogoriella | Brevibacterium | vietnamiensis |
| | mobilis | caseilytica | frigoritolerans | Buttiauxella |
| | Belliella | Bordetella | Brevibacterium | Buttiauxella |
| | Belliella baltica | Bordetella avium | halotolerans | agrestis |
| | Bellilinea | Bordetella | Brevibacterium | Buttiauxella |
| | Bellilinea | bronchiseptica | iodinum | brennerae |
| | caldifistulae | Bordetella hinzii | Brevibacterium | Buttiauxella |
| | Belnapia | Bordetella holmesii | linens | ferragutiae |
| | Belnapia | Bordetella parapertussis | Brevibacterium | Buttiauxella |
| | moabensis | Bordetella pertussis | lyticum | gaviniae |
| | Bergeriella | Bordetella petrii | Brevibacterium | Buttiauxella |
| | Bergeriella | Bordetella trematum | mcbrellneri | izardii |
| | denitrificans | | Brevibacterium | Buttiauxella |
| | Beutenbergia | | otitidis | noackiae |
| | Beutenbergia | | Brevibacterium | Buttiauxella |
| | cavernae | | oxydans | warmboldiae |
| | | | Brevibacterium | Butyrivibrio |
| | | | paucivorans | Butyrivibrio |
| | | | Brevibacterium | fibrisolvens |
| | | | stationis | Butyrivibrio |
| | | | | hungatei |
| | | | | Butyrivibrio |
| | | | | proteoclasticus |

| | | | | |
|---|---|---|---|---|
| Bacillus | | | | |
| B. acidiceler | B. aminovorans | B. glucanolyticus | B. taeanensis | B. lautus |
| B. acidicola | B. amylolyticus | B. gordonae | B. tequilensis | B. lehensis |
| B. acidiproducens | B. andreesenii | B. gottheilii | B. thermantarcticus | B. lentimorbus |
| B. acidocaldarius | B. aneurinilyticus | B. graminis | B. thermoaerophilus | B. lentus |
| B. acidoterrestris | B. anthracis | B. halmapalus | B. thermoamylovorans | B. licheniformis |
| B. aeolius | B. aquimaris | B. haloalkaliphilus | B. thermocatenulatus | B. ligniniphilus |
| B. aerius | B. arenosi | B. halochares | B. thermocloacae | B. litoralis |
| B. aerophilus | B. arseniciselenatis | B. halodenitrificans | B. thermocopriae | B. locisalis |
| B. agaradhaerens | B. arsenicus | B. halodurans | B. thermodenitrificans | B. luciferensis |
| B. agri | B. aurantiacus | B. halophilus | B. thermoglucosidasius | B. luteolus |
| B. aidingensis | B. arvi | B. halosaccharovorans | B. thermolactis | B. luteus |
| B. akibai | B. aryabhattai | B. hemicellulosilyticus | B. thermoleovorans | B. macauensis |
| B. alcalophilus | B. asahii | B. hemicentroti | B. thermophilus | B. macerans |
| B. algicola | B. atrophaeus | B. herbersteinensis | B. thermoruber | B. macquariensis |
| B. alginolyticus | B. axarquiensis | B. horikoshii | B. thermosphaericus | B. macyae |
| B. alkalidiazotrophicus | B. azotofixans | B. horneckiae | B. thiaminolyticus | B. malacitensis |
| B. alkalinitrilicus | B. azotoformans | B. horti | B. thioparans | B. mannanilyticus |
| B. alkalisediminis | B. badius | B. huizhouensis | B. thuringiensis | B. marisflavi |
| B. alkalitelluris | B. barbaricus | B. humi | B. tianshenii | B. marismortui |
| B. altitudinis | B. bataviensis | B. hwajinpoensis | B. trypoxylicola | B. marmarensis |
| B. alveayuensis | B. beijingensis | B. idriensis | B. tusciae | B. massiliensis |
| B. alvei | B. benzoevorans | B. indicus | B. validus | B. megaterium |
| B. amyloliquefaciens | B. beringensis | B. infantis | B. vallismortis | B. mesonae |
| B. a. subsp. amyloliquefaciens | B. berkeleyi | B. infernus | B. vedderi | B. methanolicus |
| B. a. subsp. plantarum | B. beveridgei | B. insolitus | B. velezensis | B. methylotrophicus |
| B. dipsosauri | B. bogoriensis | B. invictae | B. vietnamensis | B. migulanus |
| B. drentensis | B. boroniphilus | B. iranensis | B. vireti | B. mojavensis |
| B. edaphicus | B. borstelensis | B. isabeliae | B. vulcani | B. mucilaginosus |
| B. ehimensis | B. brevis Migula | B. isronensis | B. wakoensis | B. muralis |
| B. eiseniae | B. butanolivorans | B. jeotgali | B. weihenstephanensis | B. murimartini |
| B. enclensis | B. canaveralius | B. kaustophilus | B. xiamenensis | B. mycoides |
| B. endophyticus | B. carboniphilus | B. kobensis | B. xiaoxiensis | B. naganoensis |
| B. endoradicis | B. cecembensis | B. kochii | B. zhanjiangensis | B. nanhaiensis |
| B. farraginis | B. cellulosilyticus | B. kokeshiiformis | B. peoriae | B. nanhaiisediminis |
| B. fastidiosus | B. centrosporus | B. koreensis | B. persepolensis | B. nealsonii |
| B. fengqiuensis | B. cereus | B. korlensis | B. persicus | B. neidei |
| B. firmus | B. chagannorensis | B. kribbensis | B. pervagus | B. neizhouensis |
| B. flexus | B. chitinolyticus | B. krulwichiae | B. plakortidis | B. niabensis |
| B. foraminis | B. chondroitinus | B. laevolacticus | B. pocheonensis | B. niacini |
| B. fordii | B. choshinensis | B. larvae | B. polygoni | B. novalis |
| B. formosus | B. chungangensis | B. laterosporus | B. polymyxa | B. oceanisediminis |
| B. fortis | B. cibi | B. salexigens | B. popilliae | B. odysseyi |
| B. fumarioli | B. circulans | B. saliphilus | B. pseudalcalophilus | B. okhensis |
| B. funiculus | B. clarkii | B. schlegelii | B. pseudofirmus | B. okuhidensis |
| B. fusiformis | B. clausii | B. sediminis | B. pseudomycoides | B. oleronius |
| B. galactophilus | B. coagulans | B. selenatarsenatis | B. psychrodurans | B. oryzaecorticis |
| B. galactosidilyticus | B. coahuilensis | B. selenitireducens | B. psychrophilus | B. oshimensis |
| B. galliciensis | B. cohnii | B. seohaeanensis | B. psychrosaccharolyticus | B. pabuli |
| B. gelatini | B. composti | B. shacheensis | B. psychrotolerans | B. pakistanensis |
| B. gibsonii | B. curdlanolyticus | B. shackletonii | B. pulvifaciens | B. pallidus |
| B. ginsengi | B. cycloheptanicus | B. siamensis | B. pumilus | B. pallidus |
| B. ginsengihumi | B. cytotoxicus | B. silvestris | B. purgationiresistens | B. panacisoli |
| B. ginsengisoli | B. daliensis | B. simplex | B. pycnus | B. panaciterrae |
| B. globisporus | B. decisifrondis | B. siralis | B. qingdaonensis | B. pantothenticus |
| (eg, B. g. subsp. | B. decolorationis | B. smithii | B. qingshengii | B. parabrevis |
| Globisporus; | B. deserti | B. soli | B. reuszeri | B. paraflexus |
| or B. g. subsp. | | B. solimangrovi | B. rhizosphaerae | B. pasteurii |
| Marinus) | | B. solisalsi | B. rigui | B. patagoniensis |
| | | B. songklensis | B. ruris | |
| | | B. sonorensis | B. safensis | |
| | | B. sphaericus | B. salarius | |
| | | B. sporothermodurans | | |
| | | B. stearothermophilus | | |
| | | B. stratosphericus | | |
| | | B. subterraneus | | |
| | | B. subtilis | | |
| | | (eg, B. s. subsp. | | |
| | | Inaquosorum; | | |
| | | or B. s. subsp. | | |
| | | Spizizeni; | | |
| | | or B. s. subsp. | | |
| | | Subtilis) | | |

| | | | | |
|---|---|---|---|---|
| Caenimonas | Campylobacter | Cardiobacterium | Catenuloplanes | Curtobacterium |
| Caenimonas koreensis | Campylobacter coli | Cardiobacterium | Catenuloplanes | Curtobacterium |
| Caldalkalibacillus | Campylobacter | hominis | atrovinosus | albidum |
| Caldalkalibacillus | concisus | Carnimonas | Catenuloplanes | Curtobacterium |
| uzonensis | Campylobacter | Carnimonas | castaneus | citreus |
| Caldanaerobacter | curvus | nigrificans | Catenuloplanes | |
| Caldanaerobacter | Campylobacter fetus | Carnobacterium | crispus | |
| subterraneus | Campylobacter | Carnobacterium | Catenuloplanes | |
| Caldanaerobius | gracilis | alterfunditum | indicus | |
| Caldanaerobius | Campylobacter | Carnobacterium | Catenuloplanes | |
| fijiensis | helveticus | divergens | japonicus | |
| Caldanaerobius | Campylobacter | Carnobacterium | Catenuloplanes | |
| polysaccharolyticus | hominis | funditum | nepalensis | |
| Caldanaerobius zeae | Campylobacter | Carnobacterium | Catenuloplanes | |
| Caldanaerovirga | hyointestinalis | gallinarum | niger | |
| Caldanaerovirga | Campylobacter | Carnobacterium | Chryseobacterium | |
| acetigignens | jejuni | maltaromaticum | Chryseobacterium | |
| Caldicellulosiruptor | Campylobacter lari | Carnobacterium | balustinum | |
| Caldicellulosiruptor | Campylobacter | mobile | Citrobacter | |
| bescii | mucosalis | Carnobacterium | C. amalonaticus | |
| Caldicellulosiruptor | Campylobacter | viridans | C. braakii | |
| kristjanssonii | rectus | Caryophanon | C. diversus | |
| Caldicellulosiruptor | Campylobacter | Caryophanon | C. farmeri | |
| owensensis | showae | latum | C. freundii | |
| | Campylobacter | Caryophanon | C. gillenii | |
| | sputorum | tenue | C. koseri | |
| | Campylobacter | Catellatospora | C. murliniae | |
| | upsaliensis | Catellatospora | C. pasteurii^{[1]} | |
| | Capnocytophaga | citrea | C. rodentium | |
| | Capnocytophaga | Catellatospora | C. sedlakii | |
| | canimorsus | methionotrophica | C. werkmanii | |
| | Capnocytophaga | Catenococcus | C. youngae | |
| | cynodegmi | Catenococcus | Clostridium | |
| | Capnocytophaga | thiocycli | (see below) | |
| | gingivalis | | Coccochloris | |
| | Capnocytophaga | | Coccochloris | |
| | granulosa | | elabens | |
| | Capnocytophaga | | Corynebacterium | |
| | haemolytica | | Corynebacterium | |
| | Capnocytophaga | | flavescens | |
| | ochracea | | Corynebacterium | |
| | Capnocytophaga | | variabile | |
| | sputigena | | | |

Clostridium
Clostridium absonum, Clostridium aceticum, Clostridium acetireducens, Clostridium acetobutylicum,
Clostridium acidisoli, Clostridium aciditolerans, Clostridium acidurici, Clostridium aerotolerans,
Clostridium aestuarii, Clostridium akagii, Clostridium aldenense, Clostridium aldrichii, Clostridium
algidicarni, Clostridium algidixylanolyticum, Clostridium algifaecis, Clostridium algoriphilum, Clostridium
alkalicellulosi, Clostridium aminophilum, Clostridium aminovalericum, Clostridium amygdalinum,
Clostridium amylolyticum, Clostridium arbusti, Clostridium arcticum, Clostridium argentinense,
Clostridium asparagiforme, Clostridium aurantibutyricum, Clostridium autoethanogenum, Clostridium
baratii, Clostridium barkeri, Clostridium bartlettii, Clostridium beijerinckii, Clostridium bifermentans,
Clostridium bolteae, Clostridium bornimense, Clostridium botulinum, Clostridium bowmanii, Clostridium
bryantii, Clostridium butyricum, Clostridium cadaveris, Clostridium caenicola, Clostridium
caminithermale, Clostridium carboxidivorans, Clostridium carnis, Clostridium cavendishii, Clostridium
celatum, Clostridium celerecrescens, Clostridium cellobioparum, Clostridium cellulofermentans,
Clostridium cellulolyticum, Clostridium cellulosi, Clostridium cellulovorans, Clostridium chartatabidum,
Clostridium chauvoei, Clostridium chromiireducens, Clostridium citroniae, Clostridium clariflavum,
Clostridium clostridioforme, Clostridium coccoides, Clostridium cochlearium, Clostridium colletant,
Clostridium colicanis, Clostridium colinum, Clostridium collagenovorans, Clostridium cylindrosporum,
Clostridium difficile, Clostridium diolis, Clostridium disporicum, Clostridium drakei, Clostridium durum,
Clostridium estertheticum, Clostridium estertheticum estertheticum, Clostridium estertheticum
laramiense, Clostridium fallax, Clostridium felsineum, Clostridium fervidum, Clostridium fimetarium,
Clostridium formicaceticum, Clostridium frigidicarnis, Clostridium frigoris, Clostridium ganghwense,
Clostridium gasigenes, Clostridium ghonii, Clostridium glycolicum, Clostridium glycyrrhizinilyticum,
Clostridium grantii, Clostridium haemolyticum, Clostridium halophilum, Clostridium hastiforme,
Clostridium hathewayi, Clostridium herbivorans, Clostridium hiranonis, Clostridium histolyticum,
Clostridium homopropionicum, Clostridium huakuii, Clostridium hungatei, Clostridium hydrogeniformans,
Clostridium hydroxybenzoicum, Clostridium hylemonae, Clostridium jejuense, Clostridium indolis,
Clostridium innocuum, Clostridium intestinale, Clostridium irregulare, Clostridium isatidis, Clostridium
josui, Clostridium kluyveri, Clostridium lactatifermentans, Clostridium lacusfryxellense, Clostridium
laramiense, Clostridium lavalense, Clostridium lentocellum, Clostridium lentoputrescens, Clostridium
leptum, Clostridium limosum, Clostridium litorale, Clostridium lituseburense, Clostridium ljungdahlii,
Clostridium lortetii, Clostridium lundense, Clostridium magnum, Clostridium malenominatum,
Clostridium mangenotii, Clostridium mayombei, Clostridium methoxybenzovorans, Clostridium
methylpentosum, Clostridium neopropionicum, Clostridium nexile, Clostridium nitrophenolicum,
Clostridium novyi, Clostridium oceanicum, Clostridium orbiscindens, Clostridium oroticum, Clostridium
oxalicum, Clostridium papyrosolvens, Clostridium paradoxum, Clostridium paraperfringens (Alias: C. welchii),
Clostridium paraputrificum, Clostridium pascui, Clostridium pasteurianum, Clostridium
peptidivorans, Clostridium perenne, Clostridium perfringens, Clostridium pfennigii, Clostridium
phytofermentans, Clostridium piliforme, Clostridium polysaccharolyticum, Clostridium populeti,
Clostridium propionicum, Clostridium proteoclasticum, Clostridium proteolyticum, Clostridium
psychrophilum, Clostridium puniceum, Clostridium purinilyticum, Clostridium putrefaciens, Clostridium
putrificum, Clostridium quercicolum, Clostridium quinii, Clostridium ramosum, Clostridium rectum,
Clostridium roseum, Clostridium saccharobutylicum, Clostridium saccharogumia, Clostridium
saccharolyticum, Clostridium saccharoperbutylacetonicum, Clostridium sardiniense, Clostridium
sartagoforme, Clostridium scatologenes, Clostridium schirmacherense, Clostridium scindens, Clostridium
septicum, Clostridium sordellii, Clostridium sphenoides, Clostridium spiroforme, Clostridium sporogenes,
Clostridium sporosphaeroides, Clostridium stercorarium, Clostridium stercorarium leptospartum,
Clostridium stercorarium stercorarium, Clostridium stercorarium thermolacticum, Clostridium sticklandii,
Clostridium straminisolvens, Clostridium subterminale, Clostridium sufflavum, Clostridium sulfidigenes,
Clostridium symbiosum, Clostridium tagluense, Clostridium tepidiprofundi, Clostridium termitidis,
Clostridium tertium, Clostridium tetani, Clostridium tetanomorphum, Clostridium thermaceticum,
Clostridium thermautotrophicum, Clostridium thermoalcaliphilum, Clostridium thermobutyricum,
Clostridium thermocellum, Clostridium thermocopriae, Clostridium thermohydrosulfuricum, Clostridium
thermolacticum, Clostridium thermopalmarium, Clostridium thermopapyrolyticum, Clostridium
thermosaccharolyticum, Clostridium thermosuccinogenes, Clostridium thermosulfurigenes, Clostridium
thiosulfatireducens, Clostridium tyrobutyricum, Clostridium uliginosum, Clostridium ultunense,
Clostridium villosum, Clostridium vincentii, Clostridium viride, Clostridium xylanolyticum, Clostridium
xylanovorans

| | | | |
|---|---|---|---|
| Dactylosporangium | Deinococcus | Delftia | Echinicola |
| Dactylosporangium | Deinococcus | Delftia | Echinicola |
| aurantiacum | aerius | acidovorans | pacifica |
| Dactylosporangium | Deinococcus | Desulfovibrio | Echinicola |
| fulvum | apachensis | Desulfovibrio | vietnamensis |
| Dactylosporangium | Deinococcus | desulfuricans | |
| matsuzakiense | aquaticus | Diplococcus | |
| Dactylosporangium | Deinococcus | Diplococcus | |
| roseum | aquatilis | pneumoniae | |
| Dactylosporangium | Deinococcus caeni | | |
| thailandense | Deinococcus | | |
| Dactylosporangium | radiodurans | | |
| vinaceum | Deinococcus | | |
| | radiophilus | | |

| | | | |
|---|---|---|---|
| Enterobacter | Enterobacter kobei | Faecalibacterium | Flavobacterium |
| E. aerogenes | E. ludwigii | Faecalibacterium | Flavobacterium |
| E. amnigenus | E. mori | prausnitzii | antarcticum |
| E. agglomerans | E. nimipressuralis | Fangia | Flavobacterium |
| E. arachidis | E. oryzae | Fangia | aquatile |
| E. asburiae | E. pulveris | hongkongensis | Flavobacterium |
| E. cancerogenous | E. pyrinus | Fastidiosipila | aquidurense |
| E. cloacae | E. radicincitans | Fastidiosipila | Flavobacterium |
| E. cowanii | E. taylorae | sanguinis | balustinum |
| E. dissolvens | E. turicensis | Fusobacterium | Flavobacterium |
| E. gergoviae | E. sakazakii | Fusobacterium | croceum |
| E. helveticus | Enterobacter soli | nucleatum | Flavobacterium |
| E. hormaechei | Enterococcus | | cucumis |
| E. intermedius | Enterococcus | | Flavobacterium |
| | durans | | daejeonense |
| | Enterococcus | | Flavobacterium |
| | faecalis | | defluvii |
| | Enterococcus | | Flavobacterium |
| | faecium | | degerlachei |
| | Erwinia | | Flavobacterium |
| | Erwinia hapontici | | denitrificans |
| | Escherichia | | Flavobacterium |
| | Escherichia coli | | filum |
| | | | Flavobacterium |
| | | | flevense |
| | | | Flavobacterium |
| | | | frigidarium |
| | | | Flavobacterium |
| | | | mizutaii |
| | | | Flavobacterium |
| | | | okeanokoites |

| | | | |
|---|---|---|---|
| Gaetbulibacter | Haemophilus | Ideonella | Janibacter |
| Gaetbulibacter | Haemophilus | Ideonella | Janibacter |
| saemankumensis | aegyptius | azotifigens | anophelis |
| Gallibacterium | Haemophilus | Idiomarina | Janibacter |
| Gallibacterium anatis | aphrophilus | Idiomarina | corallicola |
| Gallicola | Haemophilus felis | abyssalis | Janibacter |
| Gallicola barnesae | Haemophilus | Idiomarina | limosus |
| Garciella | gallinariim | baltica | Janibacter |
| Garciella | Haemophilus | Idiomarina | melonis |
| nitratireducens | haemolyticus | fontislapidosi | Janibacter |
| Geobacillus | Haemophilus | Idiomarina | terrae |
| Geobacillus | influenzae | loihiensis | Jannaschia |
| thermoglucosidasius | Haemophilus | Idiomarina | Jannaschia |
| Geobacillus | paracuniculus | ramblicola | cystaugens |
| stearothermophilus | Haemophilus | Idiomarina | Jannaschia |
| Geobacter | parahaemolyticus | seosinensis | helgolandensis |
| Geobacter | Haemophilus | Idiomarina | Jannaschia |
| bemidjiensis | parainfluenzae | zobellii | pohangensis |
| Geobacter brememis | Haemophilus | Ignatzschineria | Jannaschia |
| Geobacter chapellei | paraphrohaemolyticus | Ignatzschineria | rubra |
| Geobacter grbiciae | Haemophilus | larvae | Janthinobacterium |
| Geobacter | parasuis | Ignavigranum | Janthinobacterium |
| hydrogenophilus | Haemophilus | Ignavigranum | agaricidamnosum |
| Geobacter lovleyi | pittmaniae | ruoffiae | Janthinobacterium |
| Geobacter | Hafnia | Ilumatobacter | lividum |
| metallireducens | Hafnia alvei | Ilumatobacter | Jejuia |
| Geobacter pelophilus | Hahella | fluminis | Jejuia |
| Geobacter pickeringii | Hahella | Ilyobacter | pallidilutea |
| Geobacter | ganghwensis | Ilyobacter | Jeotgalibacillus |
| sulfurreducens | Halalkalibacillus | delafieldii | Jeotgalibacillus |
| Geodermatophilus | Halalkalibacillus | Ilyobacter | alimentarius |
| Geodermatophilus | halophilus | insuetus | Jeotgalicoccus |
| obscurus | Helicobacter | Ilyobacter | Jeotgalicoccus |
| Gluconacetobacter | Helicobacter | polytropus | halotolerans |
| Gluconacetobacter | pylori | Ilyobacter | |
| xylinus | | tartaricus | |
| Gordonia | | | |
| Gordonia | | | |
| rubripertincta | | | |

| | | | | |
|---|---|---|---|---|
| Kaistia | Labedella | Listeria ivanovii | Micrococcus | Nesterenkonia |
| Kaistia adipata | Labedella | L. marthii | Micrococcus | Nesterenkonia |
| Kaistia soli | gwakjiensis | L. monocytogenes | luteus | holobia |
| Kangiella | Labrenzia | L. newyorkensis | Micrococcus | Nocardia |
| Kangiella | Labrenzia | L. riparia | lylae | Nocardia |
| aquimarina | aggregata | L. rocourtiae | Moraxella | argentinensis |
| Kangiella koreensis | Labrenzia alba | L. seeligeri | Moraxella bovis | Nocardia |
| Kerstersia | Labrenzia | L. weihenstephanensis | Moraxella | corallina |
| Kerstersia gyiorum | alexandrii | L. welshimeri | nonliquefaciens | Nocardia |
| Kiloniella | Labrenzia marina | Listonella | Moraxella | otitidiscaviarum |
| Kiloniella laminariae | Labrys | Listonella | osloensis | |
| Klebsiella | Labrys | anguillarum | Nakamurella | |
| K. granulomatis | methylaminiphilus | Macrococcus | Nakamurella | |
| K. oxytoca | Labrys | Macrococcus | multipartita | |
| K. pneumoniae | miyagiensis | bovicus | Nannocystis | |
| K. terrigena | Labrys monachus | Marinobacter | Nannocystis | |
| K. variicola | Labrys | Marinobacter | pusilla | |
| Kluyvera | okinawensis | algicola | Natranaerobius | |
| Kluyvera ascorbata | Labrys | Marinobacter | Natranaerobius | |
| Kocuria | portucalensis | bryozoorum | thermophilus | |
| Kocuria roasea | Lactobacillus | Marinobacter | Natranaerobius | |
| Kocuria varians | [see below] | flavimaris | trueperi | |
| Kurthia | Laceyella | Meiothermus | Naxibacter | |
| Kurthia zopfii | Laceyella putida | Meiothermus | Naxibacter | |
| | Lechevalieria | ruber | alkalitolerans | |
| | Lechevalieria | Methylophilus | Neisseria | |
| | aerocolonigenes | Methylophilus | Neisseria cinerea | |
| | Legionella | methylotrophus | Neisseria | |
| | [see below] | Microbacterium | denitrificans | |
| | Listeria | Microbacterium | Neisseria | |
| | L. aquatica | ammoniaphilum | gonorrhoeae | |
| | L. booriae | Microbacterium | Neisseria | |
| | L. cornellensis | arborescens | lactamica | |
| | L. fleischmannii | Microbacterium | Neisseria mucosa | |
| | L. floridensis | liquefaciens | Neisseria sicca | |
| | L. grandensis | Microbacterium | Neisseria subflava | |
| | L. grayi | oxydans | Neptunomonas | |
| | L. innocua | | Neptunomonas | |
| | | | japonica | |

| | | | | |
|---|---|---|---|---|
| Lactobacillus | | | | |
| L. acetotolerans | L. catenaformis | L. mali | L. parakefiri | L. sakei |
| L. acidifarinae | L. ceti | L. manihotivorans | L. paralimentarius | L. salivarius |
| L. acidipiscis | L. coleohominis | L. mindensis | L. paraplantarum | L. sanfranciscensis |
| L. acidophilus | L. collinoides | L. mucosae | L. pentosus | L. satsumensis |
| Lactobacillus agilis | L. composti | L. murinus | L. perolens | L. secaliphilus |
| L. algidus | L. concavus | L. nagelii | L. plantarum | L. sharpeae |
| L. alimentarius | L. coryniformis | L. namurensis | L. pontis | L. siliginis |
| L. amylolyticus | L. crispatus | L. nantensis | L. protectus | L. spicheri |
| L. amylophilus | L. crustorum | L. oligofermentans | L. psittaci | L. suebicus |
| L. amylotrophicus | L. curvatus | L. oris | L. rennini | L. thailandensis |
| L. amylovorus | L. delbrueckii | L. panis | L. reuteri | L. ultunensis |
| L. animalis | subsp. bulgaricus | L. pantheris | L. rhamnosus | L. vaccinostercus |
| L. antri | L. delbrueckii | L. parabrevis | L. rimae | L. vaginalis |
| L. apodemi | subsp. delbrueckii | L. parabuchneri | L. rogosae | L. versmoldensis |
| L. aviarius | L. delbrueckii | L. paracasei | L. rossiae | L. vini |
| L. bifermentans | subsp. lactis | L. paracollinoides | L. ruminis | L. vitulinus |
| L. brevis | L. dextrinicus | L. parafarraginis | L. saerimneri | L. zeae |
| L. buchneri | L. diolivorans | L. homohiochii | L. jensenii | L. zymae |
| L. camelliae | L. equi | L. iners | L. johnsonii | L. gastricus |
| L. casei | L. equigenerosi | L. ingluviei | L. kalixensis | L. ghanensis |
| L. kitasatonis | L. farraginis | L. intestinalis | L. kefiranofaciens | L. graminis |
| L. kunkeei | L. farciminis | L. fuchuensis | L. kefiri | L. hammesii |
| L. leichmannii | L. fermentum | L. gallinarum | L. kimchii | L. hamsteri |
| L. lindneri | L. fornicalis | L. gasseri | L. helveticus | L. harbinensis |
| L. malefermentans | L. fructivorans | | L. hilgardii | L. hayakitensis |
| | L. frumenti | | | |

| | | | |
|---|---|---|---|
| Legionella | | | |
| Legionella | Legionella | Candidatus | Legionella |
| adelaidensis | drancourtii | Legionella jeonii | quinlivanii |
| Legionella anisa | Legionella | Legionella | Legionella |
| Legionella | dresdenensis | jordanis | rowbothamii |
| beliardensis | Legionella | Legionella | Legionella |
| Legionella | drozanskii | lansingensis | rubrilucens |
| birminghamensis | Legionella | Legionella | Legionella |
| Legionella | dumoffii | londiniensis | sainthelensi |
| bozemanae | Legionella erythra | Legionella | Legionella |
| Legionella brunensis | Legionella | longbeachae | santicrucis |
| Legionella | fairfieldensis | Legionella lytica | Legionella |
| busanensis | Legionella fallonii | Legionella | shakespearei |
| Legionella cardiaca | Legionella feeleii | maceachernii | Legionella |
| Legionella cherrii | Legionella | Legionella | spiritensis |
| Legionella | geestiana | massiliensis | Legionella |
| cincinnatiensis | Legionella | Legionella | steelei |
| Legionella | genomospecies | micdadei | Legionella |
| clemsonensis | Legionella | Legionella | steigerwaltii |
| Legionella | gormanii | monrovica | Legionella |
| donaldsonii | Legionella | Legionella | taurinensis |
| | gratiana | moravica | Legionella |
| | Legionella | Legionella | tucsonensis |
| | gresilensis | nagasakiensis | Legionella |
| | Legionella | Legionella | tunisiensis |
| | hackeliae | nautarum | Legionella |
| | Legionella | Legionella | wadsworthii |
| | impletisoli | norrlandica | Legionella |
| | Legionella | Legionella | waltersii |
| | israelensis | oakridgensis | Legionella |
| | Legionella | Legionella | worsleiensis |
| | jamestowniensis | parisiensis | Legionella |
| | | Legionella | yabuuchiae |
| | | pittsburghensis | |
| | | Legionella | |
| | | pneumophila | |
| | | Legionella | |
| | | quateirensis | |

| | | | |
|---|---|---|---|
| Oceanibulbus | Paenibacillus | Prevotella | Quadrisphaera |
| Oceanibulbus | Paenibacillus | Prevotella | Quadrisphaera |
| indolifex | thiaminolyticus | albensis | granulorum |
| Oceanicaulis | Pantoea | Prevotella amnii | Quatrionicoccus |
| Oceanicaulis | Pantoea | Prevotella | Quatrionicoccus |
| alexandrii | agglomerans | bergensis | australiensis |
| Oceanicola | Paracoccus | Prevotella bivia | Quinella |
| Oceanicola batsensis | Paracoccus | Prevotella brevis | Quinella ovalis |
| Oceanicola | alcaliphilus | Prevotella | Ralstonia |
| granulosus | Paucimonas | bryantii | Ralstonia |
| Oceanicola | Paucimonas | Prevotella buccae | eutropha |
| nanhaiensis | lemoignei | Prevotella | Ralstonia |
| Oceanimonas | Pectobacterium | buccalis | insidiosa |
| Oceanimonas | Pectobacterium | Prevotella copri | Ralstonia |
| baumannii | aroidearum | Prevotella | mannitolilytica |
| Oceaniserpentilla | Pectobacterium | dentalis | Ralstonia |
| Oceaniserpentilla | atrosepticum | Prevotella | pickettii |
| haliotis | Pectobacterium | denticola | Ralstonia |
| Oceanisphaera | betavasculorum | Prevotella disiens | pseudosolanacearum |
| Oceanisphaera | Pectobacterium | Prevotella | Ralstonia syzygii |
| donghaensis | cacticida | histicola | Ralstonia |
| Oceanisphaera | Pectobacterium | Prevotella | solanacearum |
| litoralis | carnegieana | intermedia | Ramlibacter |
| Oceanithermus | Pectobacterium | Prevotella | Ramlibacter |
| Oceanithermus | carotovorum | maculosa | henchirensis |
| desulfurans | Pectobacterium | Prevotella | Ramlibacter |
| Oceanithermus | chrysanthemi | marshii | tataouinensis |
| profundus | Pectobacterium | Prevotella | Raoultella |
| Oceanobacillus | cypripedii | melaninogenica | Raoultella |
| Oceanobacillus caeni | Pectobacterium | Prevotella micans | ornithinolytica |
| Oceanospirillum | rhapontici | Prevotella | Raoultella |
| Oceanospirillum | Pectobacterium | multiformis | planticola |
| linum | wasabiae | Prevotella | Raoultella |
| | Planococcus | nigrescens | terrigena |
| | Planococcus | Prevotella oralis | Rathayibacter |
| | citreus | Prevotella oris | Rathayibacter |
| | Planomicrobium | Prevotella | caricis |
| | Planomicrobium | oulorum | Rathayibacter |
| | okeanokoites | Prevotella pallens | festucae |
| | Plesiomonas | Prevotella salivae | Rathayibacter |
| | Plesiomonas | Prevotella | iranicus |
| | shigelloides | stercorea | Rathayibacter |
| | Proteus | Prevotella | rathayi |
| | Proteus vulgaris | tannerae | Rathayibacter |
| | | Prevotella | toxicus |
| | | timonensis | Rathayibacter |
| | | Prevotella | tritici |
| | | veroralis | Rhodobacter |
| | | Providencia | Rhodobacter |
| | | Providencia | sphaeroides |
| | | stuartii | Ruegeria |
| | | Pseudomonas | Ruegeria |
| | | Pseudomonas | gelatinovorans |
| | | aeruginosa | |
| | | Pseudomonas | |
| | | alcaligenes | |
| | | Pseudomonas | |
| | | anguillispetica | |
| | | Pseudomonas | |
| | | fluorescens | |
| | | Pseudoalteromonas | |
| | | haloplanktis | |
| | | Pseudomonas | |
| | | mendocina | |
| | | Pseudomonas | |
| | | pseudoalcaligenes | |
| | | Pseudomonas | |
| | | putida | |
| | | Pseudomonas | |
| | | tutzeri | |
| | | Pseudomonas | |
| | | syringae | |
| | | Psychrobacter | |
| | | Psychrobacter | |
| | | faecalis | |
| | | Psychrobacter | |
| | | phenylpyruvicus | |

| | | | | |
|---|---|---|---|---|
| Saccharococcus | Sagittula | Sanguibacter | Stenotrophomonas | Tatlockia |
| Saccharococcus | Sagittula stellata | Sanguibacter | Stenotrophomonas | Tatlockia |
| thermophilus | Salegentibacter | keddieii | maltophilia | maceachernii |
| Saccharomonospora | Salegentibacter | Sanguibacter | Streptococcus | Tatlockia |
| Saccharomonospora | salegens | suarezii | [also see below] | micdadei |
| azurea | Salimicrobium | Saprospira | Streptomyces | Tenacibaculum |
| Saccharomonospora | Salimicrobium | Saprospira | Streptomyces | Tenacibaculum |
| cyanea | album | grandis | achromogenes | amylolyticum |
| Saccharomonospora | Salinibacter | Sarcina | Streptomyces | Tenacibaculum |
| viridis | Salinibacter ruber | Sarcina maxima | cesalbus | discolor |
| Saccharophagus | Salinicoccus | Sarcina ventriculi | Streptomyces | Tenacibaculum |
| Saccharophagus | Salinicoccus | Sebaldella | cescaepitosus | gallaicum |
| degradans | alkaliphilus | Sebaldella | Streptomyces | Tenacibaculum |
| Saccharopolyspora | Salinicoccus | termitidis | cesdiastaticus | lutimaris |
| Saccharopolyspora | hispanicus | Serratia | Streptomyces | Tenacibaculum |
| erythraea | Salinicoccus roseus | Serratia fonticola | cesexfoliatus | mesophilum |
| Saccharopolyspora | Salinispora | Serratia | Streptomyces | Tenacibaculum |
| gregorii | Salinispora | marcescens | fimbriatus | skagerrakense |
| Saccharopolyspora | arenicola | Sphaerotilus | Streptomyces | Tepidanaerobacter |
| hirsuta | Salinispora tropica | Sphaerotilus | fradiae | Tepidanaerobacter |
| Saccharopolyspora | Salinivibrio | natans | Streptomyces | syntrophicus |
| hordei | Salinivibrio | Sphingobacterium | fulvissimus | Tepidibacter |
| Saccharopolyspora | costicola | Sphingobacterium | Streptomyces | Tepidibacter |
| rectivirgula | Salmonella | multivorum | griseoruber | formicigenes |
| Saccharopolyspora | Salmonella bongori | Staphylococcus | Streptomyces | Tepidibacter |
| spinosa | Salmonella enterica | [see below] | griseus | thalassicus |
| Saccharopolyspora | Salmonella | | Streptomyces | Thermus |
| taberi | subterranea | | lavendulae | Thermus |
| Saccharothrix | Salmonella typhi | | Streptomyces | aquaticus |
| Saccharothrix | | | phaeochromogenes | Thermus |
| australiensis | | | Streptomyces | filiformis |
| Saccharothrix | | | thermodiastaticus | Thermus |
| coeruleofusca | | | Streptomyces | thermophilus |
| Saccharothrix | | | tubercidicus | |
| espanaensis | | | | |
| Saccharothrix | | | | |
| longispora | | | | |
| Saccharothrix mutabilis | | | | |
| Saccharothrix syringae | | | | |
| Saccharothrix tangerinus | | | | |
| Saccharothrix texasensis | | | | |

| | | | |
|---|---|---|---|
| Staphylococcus | | | |
| S. arlettae | S. equorum | S. microti | S. schleiferi |
| S. agnetis | S. felis | S. muscae | S. sciuri |
| S. aureus | S. fleurettii | S. nepalensis | S. simiae |
| S. auricularis | S. gallinarum | S. pasteuri | S. simulans |
| S. capitis | S. haemolyticus | S. petrasii | S. stepanovicii |
| S. caprae | S. hominis | S. pettenkoferi | S. succinus |
| S. carnosus | S. hyicus | S. piscifermentans | S. vitulinus |
| S. caseolyticus | S. intermedius | S. pseudintermedius | S. warneri |
| S. chromogenes | S. kloosii | S. pseudolugdunensis | S. xylosus |
| S. cohnii | S. leei | S. pulvereri | |
| S. condimenti | S. lentus | S. rostri | |
| S. delphini | S. lugdunensis | S. saccharolyticus | |
| S. devriesei | S. lutrae | S. saprophyticus | |
| S. epidermidis | S. lyticans | | |
| | S. massiliensis | | |

| | | | |
|---|---|---|---|
| Streptococcus | | | |
| Streptococcus | Streptococcus | Streptococcus | Streptococcus |
| agalactiae | infantarius | orisratti | thermophilus |
| Streptococcus anginosus | Streptococcus iniae | Streptococcus | Streptococcus |
| Streptococcus bovis | Streptococcus | parasanguinis | sanguinis |
| Streptococcus canis | intermedius | Streptococcus | Streptococcus |
| Streptococcus | Streptococcus | peroris | sobrinus |
| constellatus | lactarius | Streptococcus | Streptococcus |
| Streptococcus downei | Streptococcus | pneumoniae | suis |
| Streptococcus | milleri | Streptococcus | Streptococcus |
| dysgalactiae | Streptococcus mitis | pseudopneumoniae | uberis |
| Streptococcus equines | Streptococcus | Streptococcus | Streptococcus |
| Streptococcus faecalis | mutans | pyogenes | vestibularis |
| Streptococcus ferus | Streptococcus oralis | Streptococcus | Streptococcus |
| | Streptococcus | ratti | viridans |
| | tigurinus | Streptococcus | Streptococcus |
| | | salivariu | zooepidemicus |

| | | | | |
|---|---|---|---|---|
| Uliginosibacterium | Vagococcus | Vibrio | Virgibacillus | Xanthobacter |
| Uliginosibacterium | Vagococcus | Vibrio aerogenes | Virgibacillus | Xanthobacter |
| gangwonense | carniphilus | Vibrio | halodenitrificans | agilis |
| Ulvibacter | Vagococcus | aestuarianus | Virgibacillus | Xanthobacter |
| Ulvibacter litoralis | elongatus | Vibrio albensis | pantothenticus | aminoxidans |
| Umezawaea | Vagococcus fessus | Vibrio | Weissella | Xanthobacter |
| Umezawaea tangerina | Vagococcus fluvialis | alginolyticus | Weissella cibaria | autotrophicus |
| Undibacterium | Vagococcus lutrae | Vibrio campbellii | Weissella confusa | Xanthobacter |
| Undibacterium pigrum | Vagococcus | Vibrio cholerae | Weissella | flavus |
| Ureaplasma | salmoninarum | Vibrio | halotolerans | Xanthobacter |
| Ureaplasma | Variovorax | cincinnatiensis | Weissella | tagetidis |
| urealyticum | Variovorax | Vibrio | hellenica | Xanthobacter |
| Ureibacillus | boronicumulans | coralliilyticus | Weissella | viscosus |
| Ureibacillus composti | Variovorax | Vibrio | kandleri | Xanthomonas |
| Ureibacillus | dokdonensis | cyclitrophicus | Weissella | Xanthomonas |
| suwonensis | Variovorax | Vibrio | koreensis | albilineans |
| Ureibacillus terrenus | paradoxus | diazotrophicus | Weissella minor | Xanthomonas |
| Ureibacillus | Variovorax soli | Vibrio fluvialis | Weissella | alfalfae |
| thermophilus | Veillonella | Vibrio furnissii | paramesenteroides | Xanthomonas |
| Ureibacillus | Veillonella atypica | Vibrio gazogenes | Weissella soli | arboricola |
| thermosphaericus | Veillonella caviae | Vibrio halioticoli | Weissella | Xanthomonas |
| | Veillonella criceti | Vibrio harveyi | thailandensis | axonopodis |
| | Veillonella dispar | Vibrio | Weissella | Xanthomonas |
| | Veillonella | ichthyoenteri | viridescens | campestris |
| | montpellierensis | Vibrio | Williamsia | Xanthomonas |
| | Veillonella parvula | mediterranei | Williamsia | citri |
| | Veillonella ratti | Vibrio | marianensis | Xanthomonas |
| | Veillonella | metschnikovii | Williamsia maris | codiaei |
| | rodentium | Vibrio mytili | Williamsia | Xanthomonas |
| | Venenivibrio | Vibrio natriegens | serinedens | cucurbitae |
| | Venenivibrio | Vibrio | Winogradskyella | Xanthomonas |
| | stagnispumantis | navarrensis | Winogradskyella | euvesicatoria |
| | Verminephrobacter | Vibrio nereis | thalassocola | Xanthomonas |
| | Verminephrobacter | Vibrio | Wolbachia | fragariae |
| | eiseniae | nigripulchritudo | Wolbachia | Xanthomonas |
| | Verrucomicrobium | Vibrio ordalii | persica | fuscans |
| | Verrucomicrobium | Vibrio orientalis | Wolinella | Xanthomonas |
| | spinosum | Vibrio | Wolinella | gardneri |
| | | parahaemolyticus | succinogenes | Xanthomonas |
| | | Vibrio pectenicida | Zobellia | hortorum |
| | | Vibrio penaeicida | Zobellia | Xanthomonas |
| | | Vibrio | galactanivorans | hyacinthi |
| | | proteolyticus | Zobellia uliginosa | Xanthomonas |
| | | Vibrio shilonii | Zoogloea | perforans |
| | | Vibrio splendidus | Zoogloea | Xanthomonas |
| | | Vibrio tubiashii | ramigera | phaseoli |
| | | Vibrio vulnificus | Zoogloea | Xanthomonas |
| | | | resiniphila | pisi |
| | | | | Xanthomonas |
| | | | | populi |
| | | | | Xanthomonas |
| | | | | theicola |
| | | | | Xanthomonas |
| | | | | translucens |
| | | | | Xanthomonas |
| | | | | vesicatoria |
| | | | | Xylella |
| | | | | Xylella |
| | | | | fastidiosa |
| | | | | Xylophilus |
| | | | | Xylophilus |
| | | | | ampelinus |

| | | | | |
|---|---|---|---|---|
| Xenophilus | Yangia | Yersinia | Zooshikella | Zobellella |
| Xenophilus azovorans | Yangia pacifica | mollaretii | Zooshikella | Zobellella |
| Xenorhabdus | Yaniella | Yersinia | ganghwensis | denitrificans |
| Xenorhabdus beddingii | Yaniella flava | philomiragia | Zunongwangia | Zobellella |
| Xenorhabdus bovienii | Yaniella | Yersinia pestis | Zunongwangia | taiwanensis |
| Xenorhabdus | halotolerans | Yersinia | profunda | Zeaxanthinibacter |
| cabanillasii | Yeosuana | pseudotuberculosis | Zymobacter | Zeaxanthinibacter |
| Xenorhabdus doucetiae | Yeosuana | Yersinia rohdei | Zymobacter | enoshimensis |
| Xenorhabdus griffiniae | aromativorans | Yersinia ruckeri | palmae | Zhihengliuella |
| Xenorhabdus hominickii | Yersinia | Yokenella | Zymomonas | Zhihengliuella |
| Xenorhabdus | Yersinia aldovae | Yokenella | Zymomonas | halotolerans |
| koppenhoeferi | Yersinia bercovieri | regensburgei | mobilis | Xylanibacterium |
| Xenorhabdus | Yersinia | Yonghaparkia | Zymophilus | Xylanibacterium |
| nematophila | enterocolitica | Yonghaparkia | Zymophilus | ulmi |
| Xenorhabdus poinarii | Yersinia | alkaliphila | paucivorans | |
| Xylanibacter | entomophaga | Zavarzinia | Zymophilus | |
| Xylanibacter oryzae | Yersinia | Zavarzinia | raffinosivorans | |
| | frederiksenii | compransoris | | |

TABLE 3
Sequences:
hypC2 is at position 20849-21064 in the pX1.0 backbone.

| Seq | | Position in |
|---|---|---|
| Name | Sequence | plasmid |
| SEQ ID | ATGCAGAATAAACCTACACCTGAAGAAGTAAAGAATGCGCGGGTTGCGGCAGGTCTTA | 20849 to 21064 |
| NO: 1 | CTCTTAAAGAAGCTGCTGATATTTTTGGTTATCAACTGAATTCCTGGCAGATGAAAGA | |
| hypC2 | AAGTGCAGGTAAGGCCAGTCGTTCTTTATCTATTGGTGAATATCAGTATTTATTGTTA | |
| sequence# | TTAGCAAATATGCATCCGTCTTACAGGCTGGTAAAAAAATAA | |
| SEQ ID | MQNKPTPEEVKNARVAAGLTLKEAADIFGYQLNSWQMKESAGKASRSLSIGEYQYLLL | |
| NO: 2 | LANMHPSYRLVKK | |
| Protein | | |
| sequence | | |
| SEQ ID | ATGCAGAATAAACCTACACCTGAAGAAGTAAAGAATGCGCGGGTTGCGGCAGGTCTTA | Complement* |
| NO: 3 | CTCTTAAAGAAGCTGCTGATATTTTTGGTTATCAACTGAATTCCTGGCAGATGAAAGA | (41318 to 41533) |
| | AAGTGCAGGTAAGGCCAGTCGTTCTTTATCTATTGGTGAATATCAGTATTTATTGTTA | |
| | TTAGCAAATATGCATCCGTCTTACAGGCTGGTAAAAAAATAA | |
| SEQ ID | ATGGATAAAGATAAGATAATTAAGAAAAACAGAGGTAATTACTCCTATGTAATCAGAA | 3200 to 32247 |
| NO: 4 | CGATGGATGAAGATGGGGATACGGTTTTTCACGTCTTAAAATATGTTAAGACGATTGA | |
| | TAAAACTAAAAGCAGGAAAACAGTAAGAAAATTGATAATGGACGAAAAACTCAACCTG | |
| | GCATCATTGATGCTTCTGGATAATGGGGTTTTGTGTGATTGTCTGACAAAAGGGAATG | |
| | AAAATGCAGAATAA | |
| SEQ ID | ATGAAGAGTAAACCAACGCCAGAACAGGTTAAAACCGCTCGCATTGCTGCTGGTCTTT | Complement* |
| NO: 5 | CTTTAAAAGAAGCCGCTGATACTTTTGGTTATCAGTTAAATTCATGGCAAATGAAAGA | (9946 to 10161) |
| | AAGTGCAGGTAAAGCAAGTCGTTCTTTATCTATTGGAGAATACGAGTATTTACTGCTG | |
| | TTAGCGAATCAACATCCAGAATATAAGATAGTGAAAAAATAG | |
| SEQ ID | ATGAAGAGTAAACCAACGCCAGAACAGGTTAAAGCCGCTCGCATTGCTGCTGGCCTTT | Complement |
| NO: 6 | CTTTAAAAGAAGCCGCTGATACTTTTGGTTATCAGTTAAATTCATGGCAAATGAAAGA | (60794 to 61009) |
| | AAGTGCAGGTAAAGCAAGTCGTTCTTTATCTATTGGAGAATACGAGTATTTACTTCTG | |
| | TTAGCGAATCAACATCCAGAATATAAGATAGTGAGAAAATAG | |
| SEQ ID | ATGAAGAGTAAACCAACACCAGAGCAAGTTAAAGCAGCTCGCATTGCTGCTGGCCTTT | 38158 to 38373 |
| NO: 7 | CTTTGAAAGAAGCTGCTGCTACTTTTGGTTATCAGCTAAATTCATGGCAGATGAAAGA | |
| | AAGCGCAGGTAAAGCAAGTCGTTCTTTATCTATTGGAGAATACGAGTATTTACTGCTG | |
| | TTAGCGAATCAACATCCAGAATACAAAATAGTAAAAAAATAG | |

#the transposon was inserted between the underlined A and G, thereby inactivating function of the gene to produce hypC2 KO.
*the sequence is on the reverse strand in the source sequence (i.e. the orientation is right to left, of the gene.

1. A conjugative plasmid that is devoid of a hypC2 nucleotide sequence or a homologue thereof.

2. The plasmid of claim 1, wherein the plasmid is devoid of SEQ ID NO: 1 or a nucleotide sequence that is at least 70% identical to SEQ ID NO: 1.

3. The plasmid of claim 1 or 2, wherein the plasmid comprises an OriT of an IncX plasmid, optionally wherein the plasmid is an IncX plasmid.

4. The plasmid of any preceding claim, wherein the plasmid is an Enterobacteriaceae plasmid, optionally wherein the plasmid is an E. coli, Klebsiella, Salmonella, Erwinia, Shigella, Pantoea, Proteus or Citrobacter plasmid.

5. The plasmid of any preceding claim, wherein the plasmid is capable of being hosted in an Enterobacteriaceae cell. optionally wherein the plasmid is capable of being hosted in an E. coli, Klebsiella, Salmonella, Erwinia, Shigella, Pantoea, Proteus or Citrobacter cell.

6. The plasmid of any preceding claim, wherein the plasmid is capable of being conjugatively transferred to an Enterobacteriaceae cell, optionally wherein the plasmid is capable of being conjugatively transferred to an E. coli, Klebsiella, Salmonella, Erwinia, Shigella, Pantoea, Proteus or Citrobacter cell.

7. A bacterial cell that comprises a conjugative plasmid, wherein the cell does not comprise a hypC2 protein or a homologue thereof.

8. The cell of claim 7, wherein the protein comprises the amino acid sequence of SEQ ID NO: 2 or an amino acid sequence that is at least 70% identical to SEQ ID NO: 2.

9. The cell of claim 7 or 8, wherein the cell is an Enterobacteriaceae cell, optionally wherein the cell is an E. coli, Klebsiella, Salmonella, Erwinia, Shigella, Pantoea, Proteus or Citrobacter cell.

10. The plasmid or cell of any preceding claim, wherein the plasmid comprises or encodes an antibacterial agent.

11. The plasmid or cell of any preceding claim, wherein the plasmid encodes a guide RNA or crRNA, optionally wherein the guide RNA or crRNA is capable of hybridising to a protospacer sequence of a target cell.

12. The plasmid or cell of claim 11, wherein the target cell is selected from an E. coli, Klebsiella, Salmonella, Erwinia, Shigella, Pantoea, Proteus or Citrobacter cell.

13. Use of plasmids according to any one of claims 1 to 6 and 10 to 12 in the manufacture of a composition comprising a first population of cells comprising the plasmids, for enhancing the frequency of plasmid conjugative transfer from the first cells to cells of a second population when first and second cells are in contact with each other and are bacterial cells.

14. A method of transferring a nucleic acid sequence of interest (NSI) from a donor cell to a recipient cell, wherein the NSI is comprised by a plasmid and the plasmid is comprised by the donor cell, the method comprising combining the cells to allow conjugative tranfer of the plasmid from the donor cell to the recipient cell, wherein the plasmid is according to any one of claims 1 to 6 and 10 to 12.

15. The method of claim 14, wherein the donor cell is selected from the group consisting of an E. coli, Klebsiella, Salmonella, Erwinia, Shigella, Pantoea, Proteus or Citrobacter cell and/or the recipient cell is selected from the group consisting of an E. coli, Klebsiella, Salmonella, Erwinia, Shigella, Pantoea, Proteus or Citrobacter cell.

16. The method of claim 14 or 15, wherein the recipient cell is an Enterobacteriaceae cell and/or the donor cell is an E. coli cell.

17. The method of any one of claims 14 to 16, wherein the NSI is or encodes an antibacterial agent, optionally wherein the agent is toxic to the recipient cell.

18. A method of treating an infection in a human or animal subject, wherein the infection is an infection of a plurality of bacterial cells (recipient cells), the method comprising
(i) administering a plurality of donor bacterial cells to the subject whereby recipient cells are combined with a donor cells, wherein each donor cell comprises a plasmid according to any one of claims 1 to 6 and 10 to 12;
(ii) and allowing conjugative transfer of plasmids from the donor cells to the recipient cells, wherein each transferred plasmid comprises a respective nucleic acid sequence of interest (NSI) that comprises or encodes an antibacterial agent or component thereof that is toxic to recipient cells, whereby recipient cells are killed or the growth or proliferation of recipient cells is inhibited.

19. A plasmid or cell of any one of claims 1 to 12 for use in a method according to claim 18.

20. The method, plasmid or cell of claim 18 or 19, wherein the method is further according to any one of claims 14 to 17.

21. The method, plasmid or cell of claim 18, 19 or 20, wherein the donor cell is an E. coli cell and the recipient cell is an E. coli, Klebsiella, Salmonella, Erwinia, Shigella, Pantoea, Proteus or Citrobacter cell.

22. A method of producing a conjugative plasmid that is devoid of a functional hypC2 nucleotide sequence or a homologue thereof, the method comprising
a) providing a first conjugative plasmid that comprises a first nucleotide sequence which is a hypC2 nucleotide sequence or a homologue thereof that is functional to express a hypC2 protein; and
b) deleting the first sequence from the first plasmid or mutating the first sequence to render the sequence non-functional for expression of said protein, thereby producing said conjugative plasmid.

23. The plasmid or cell of any one of claims 1 to 12 and 21, wherein said plasmid is obtainable or obtained by the method of claim 22.


SEQUENCE LISTING (7 sequences)

SEQ ID NO: 1 - DNA, 216 bp, Artificial Sequence
  hypC2 sequence
atgcagaataaacctacacctgaagaagtaaagaatgcgcgggttgcggcaggtcttactcttaaagaagctgctgatatttttggttatcaactgaattcctggcagatgaaagaaagtgcaggtaaggccagtcgttctttatctattggtgaatatcagtatttattgttattagcaaatatgcatccgtcttacaggctggtaaaaaaataa

SEQ ID NO: 2 - protein, 71 aa, Artificial Sequence
  hypC2 protein
MQNKPTPEEVKNARVAAGLTLKEAADIFGYQLNSWQMKESAGKASRSLSIGEYQYLLLLANMHPSYRLVKK

SEQ ID NO: 3 - DNA, 216 bp, Artificial Sequence; identical to SEQ ID NO: 1
  hypC2 sequence

SEQ ID NO: 4 - DNA, 246 bp, Artificial Sequence
  hypC2 sequence
atggataaagataagataattaagaaaaacagaggtaattactcctatgtaatcagaacgatggatgaagatggggatacggtttttcacgtcttaaaatatgttaagacgattgataaaactaaaagcaggaaaacagtaagaaaattgataatggacgaaaaactcaacctggcatcattgatgcttctggataatggggttttgtgtgattgtctgacaaaagggaatgaaaatgcagaataa

SEQ ID NO: 5 - DNA, 216 bp, Artificial Sequence
  hypC2 sequence
atgaagagtaaaccaacgccagaacaggttaaaaccgctcgcattgctgctggtctttctttaaaagaagccgctgatacttttggttatcagttaaattcatggcaaatgaaagaaagtgcaggtaaagcaagtcgttctttatctattggagaatacgagtatttactgctgttagcgaatcaacatccagaatataagatagtgaaaaaatag

SEQ ID NO: 6 - DNA, 216 bp, Artificial Sequence
  hypC2 sequence
atgaagagtaaaccaacgccagaacaggttaaagccgctcgcattgctgctggcctttctttaaaagaagccgctgatacttttggttatcagttaaattcatggcaaatgaaagaaagtgcaggtaaagcaagtcgttctttatctattggagaatacgagtatttacttctgttagcgaatcaacatccagaatataagatagtgagaaaatag

SEQ ID NO: 7 - DNA, 216 bp, Artificial Sequence
  hypC2 sequence
atgaagagtaaaccaacaccagagcaagttaaagcagctcgcattgctgctggcctttctttgaaagaagctgctgctacttttggttatcagctaaattcatggcagatgaaagaaagcgcaggtaaagcaagtcgttctttatctattggagaatacgagtatttactgctgttagcgaatcaacatccagaatacaaaatagtaaaaaaatag